(19) United States
(12) Patent Application Publication
(10) Pub. No.: US 2025/0249007 A1
(43) Pub. Date: Aug. 7, 2025

(54) PHARMACEUTICAL COMPOSITIONS OF ENSIFENTRINE FOR CHRONIC OBSTRUCTIVE PULMONARY DISEASE

(71) Applicant: Verona Pharma PLC, Cardiff (GB)

(72) Inventors: Peter Lionel SPARGO, Canterbury (GB); Tara Renae RHEAULT, Cary, NC (US); Doris K. WEILERT, Lee's Summit, MO (US); Kathleen RICKARD, Durham, NC (US); Thomas BENGTSSON, Lund (SE); Kevin TURNER, Leeds (GB)

(73) Assignee: Verona Pharma PLC, Cardiff (GB)

(21) Appl. No.: 19/046,710

(22) Filed: Feb. 6, 2025

Related U.S. Application Data
(60) Provisional application No. 63/550,792, filed on Feb. 7, 2024.

(51) Int. Cl. A61K 31/519; A61P 11/00

(52) U.S. Cl. CPC A61K 31/519; A61P 11/00

(57) ABSTRACT

Provided herein are pharmaceutical compositions and methods for providing blood plasma concentrations of ensifentrine to specific patient group populations including those with modified renal or hepatic impairment.

This application claims the benefit of U.S. Provisional Application No. 63/550,792, filed Feb. 7, 2024, which is incorporated herein by reference in its entirety.

### BACKGROUND

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

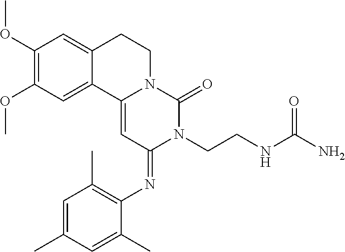 [SMILES: COc1cc2c(cc1OC)-c1c/c(=N\c3c(C)cc(C)cc3C)n(CCNC(N)=O)c(=O)n1CC2]

COPD is a progressive, long-term condition and can be experienced by patients at different levels of severity. The symptoms (e.g., breathlessness and cough) of COPD can range from mild to severe, and different treatment options can be preferred for different severities of COPD. Treatment of COPD typically comprises maintenance treatment in which a subject is administered a drug on a regular basis (e.g., once or twice daily) to improve lung function and ameliorate the symptoms of COPD.

### SUMMARY

Provided herein are pharmaceutical compositions and methods for treating COPD. In some instances, the methods provided herein comprise treatment of specific subgroup populations of COPD subjects including subjects with varying renal function or hepatic function, who may experience modified exposure and clearance of ensifentrine. About 15% of US adults are estimated to have chronic kidney disease (e.g., about 37 million people). More than 100 million people in the US have some form of liver disease (e.g., hepatic dysfunction). Given the large population of individuals with modified renal or hepatic function, it would be desirable to provide a blood plasma concentration of ensifentrine having a desirable pharmacokinetic profile to subjects who experience modified renal function or hepatic function (e.g., such as a decrease in function of either). Provided herein are methods of providing blood plasma concentrations of ensifentrine with desirable pharmacokinetic profiles for subjects with normal renal impairment to mild renal impairment as well as for subjects with moderate renal impairment. Also provided herein are methods of providing blood plasma concentrations of ensifentrine with desirable pharmacokinetic profiles for subjects with healthy liver function, moderate hepatic impairment, or severe hepatic impairment. The development of methods to treat subjects with modified renal impairment or modified hepatic impairment, is desirable to achieve dosage forms suitable for said patient groups.

In some embodiments, provided herein is a method of treating chronic obstructive pulmonary disease (COPD) in a human subject having moderate renal impairment, the method comprising administering to the human subject a liquid pharmaceutical composition comprising a therapeutically effective amount of ensifentrine or a pharmaceutically acceptable salt thereof. In some embodiments, the human subject has an absolute estimated glomerular filtration rate (aEGFR) of less than 60 mL/min. In some embodiments, the liquid pharmaceutical composition provides a blood plasma concentration of ensifentrine after administration by inhalation to the human subject, the blood plasma concentration comprising a mean Cₘₐₓ of from about 500 pg/mL to about 700 pg/mL. In some embodiments, the blood plasma concentration comprises a mean Cₘₐₓ of from about 550 pg/mL to about 650 pg/mL. In some embodiments, the blood plasma concentration comprises a mean Cₘₐₓ of about 604 pg/mL. In some embodiments, the Cₘₐₓ is at least 10% higher than a Cₘₐₓ for a subject administered the liquid pharmaceutical composition by inhalation and not having moderate renal impairment. In some embodiments, the blood plasma concentration comprises a mean AUC₀₋ₜₐᵤ of from about 2600 pg/mL*h to about 3600 pg/mL*h. In some embodiments, the blood plasma concentration comprises a mean AUC₀₋ₜₐᵤ of from about 3000 pg/mL*h to about 3400 pg/mL*h. In some embodiments, the blood plasma concentration comprises a mean AUC₀₋ₜₐᵤ of about 3200 pg/mL*h. In some embodiments, the mean AUC₀₋ₜₐᵤ is at least 25% higher than a mean AUC₀₋ₜₐᵤ for a subject administered the liquid pharmaceutical composition by inhalation and not having moderate renal impairment. In some embodiments, the blood plasma concentration comprises a mean Tₘₐₓ of from about 1 h to 2 hs. In some embodiments, the blood plasma concentration comprises a mean Tₘₐₓ of from about 1.2 h to about 1.7 h. In some embodiments, the mean Cₘₐₓ, mean AUC₀₋ₜₐᵤ, and mean Tₘₐₓ are measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h) after administration of the liquid pharmaceutical composition.

In some embodiments, provided herein is a method of treating chronic obstructive pulmonary disease (COPD) in a human subject with a liver function comprising healthy liver function, moderate hepatic impairment, or severe hepatic impairment, the method comprising administering to the human subject a liquid pharmaceutical composition comprising a therapeutically effective amount of ensifentrine or a pharmaceutically acceptable salt thereof, wherein the liquid pharmaceutical composition provides a blood plasma concentration of ensifentrine comprising an AUCt,ss which is substantially the same for subjects comprising healthy liver function, moderate hepatic impairment, or severe hepatic impairment. In some embodiments, the mean AUC_{t,ss} is of from about 5000 pg/mL*h to about 10000 pg/mL*h. In some embodiments, the mean AUC_{t,ss} is of from about 5000 pg/mL*h to about 7500 pg/mL*h. In some embodiments, the liver function of the human subject is classified using Child Pugh score. In some embodiments, the administering is by inhalation. In some embodiments, the inhalation is by nebulizer. In some embodiments, the ensifentrine is ensifentrine free base. In some embodiments, the administering to the human subject is at least once, twice, or three times per day. In some embodiments, the administering to the human subject is at least twice a day. In some embodiments, the therapeutically effective amount of ensifentrine or the pharmaceutically acceptable salt thereof is of from about 2 mg to about 4 mg. In some embodiments, the therapeutically effective amount of ensifentrine or the pharmaceutically acceptable salt thereof is 3 mg. In some embodiments, the therapeutically effective amount of ensifentrine or the pharmaceutically acceptable salt thereof comprises a total daily dose of the ensifentrine or the pharmaceutically acceptable salt thereof of from about 0.5 mg to about 10 mg. In some embodiments, the total daily dose is of from about 5 mg to about 7 mg. In some embodiments, the administering to the human subject comprises a first dose and a second dose per day. In some embodiments, the first dose comprises from about 2 mg to about 4 mg of the ensifentrine or the pharmaceutically acceptable salt thereof and the second dose comprises of from about 2 mg to about 4 mg of ensifentrine or the pharmaceutically acceptable salt thereof. In some embodiments, the first dose and the second dose comprise about 3 mg of the ensifentrine or the pharmaceutically acceptable salt thereof. In some embodiments, the first dose is administered in the morning and the second dose is administered in the evening. In some embodiments, the first dose is administered within three hours after the human subject waking, and the second dose is administered within three hours before the human subject sleeps. In some embodiments, the first dose and the second dose are administered to the human subject about 10 h to about 14 h apart. In some embodiments, the administering to the human subject is twice per day for at least 8 weeks. In some embodiments, the administering to the human subject is twice per day for at least 16 weeks. In some embodiments, the administering to the human subject is twice per day for at least 24 weeks. In some embodiments, the liquid pharmaceutical composition comprises a suspension of ensifentrine particles comprising the ensifentrine or a pharmaceutically acceptable salt thereof in a diluent. In some embodiments, the ensifentrine particles comprise at least 95% by weight of the ensifentrine or the pharmaceutically acceptable salt thereof. In some embodiments, the ensifentrine particles comprise at least 95% by weight of ensifentrine free base. In some embodiments, the ensifentrine is in the form of a crystalline solid consisting of greater than 99% by weight of ensifentrine free base, at least 95% in the polymorphic form of a thermodynamically stable polymorph (I) of ensifentrine free base having the following structural parameters obtained by single crystal analysis shown in Table 1.

TABLE 1

| Wavelength | 0.71073 Å | |
|---|---|---|
| Crystal system | Triclinic | |
| Space group | P-1 | |
| Unit cell dimensions | a = 8.1246(4) Å | α = 91.583(4)° |
| | b = 11.4573(5) Å | β = 90.299(4)° |
| | c = 13.2398(6) Å | γ = 99.628(4)° |
| Volume | 1214.56(10) Å³ | |
| Z | 2 | |

In some embodiments, the ensifentrine particles comprise from about 0% to about 0.6% by weight of 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU) relative to total weight of the ensifentrine or the pharmaceutically acceptable salt thereof. In some embodiments, the ensifentrine particles comprise from about 0% to about 0.5% by weight of a biuret impurity of formula (A) relative to total weight of the ensifentrine or the pharmaceutically acceptable salt thereof:

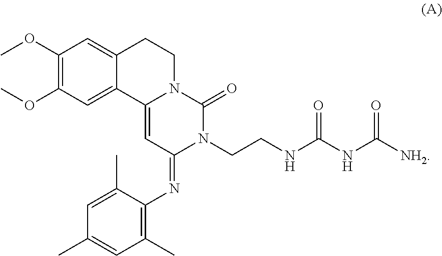 [SMILES: COc1cc2c(cc1OC)-c1c/c(=N\c3c(C)cc(C)cc3C)n(CCNC(=O)NC(N)=O)c(=O)n1CC2]

In some embodiments, the ensifentrine particles comprise from about 0% to about 0.3% by weight of the biuret impurity relative to the total weight of the ensifentrine of the pharmaceutically acceptable salt thereof. In some embodiments, the ensifentrine particles comprise from about 0.01% to about 0.3% by weight of the BMIQU relative to the total weight of the ensifentrine or the pharmaceutically acceptable salt thereof. In some embodiments, the liquid pharmaceutical composition comprises: (a) ensifentrine particles; (b) a buffer; (c) one or more surfactants; and (d) a tonicity adjuster. In some embodiments, the liquid pharmaceutical composition comprises: (a) ensifentrine particles at a concentration of from about 1 mg/mL to about 1.4 mg/mL; (b) a buffer at a concentration of from about 1 mg/mL to about 2 mg/mL; (c) one or more surfactants at a total concentration of from about 0.01 mg/mL to about 0.8 mg/mL; and (d) a tonicity adjuster at a concentration of from about 5 mg/mL to about 10 mg/mL. In some embodiments, the liquid pharmaceutical composition comprises: (a) ensifentrine particles at a concentration of from about 1 mg/mL to about 1.4 mg/mL; (b) polysorbate 20 (Tween 20) at a concentration of from about 0.3 mg/mL to about 0.7 mg/mL; (c) sorbitan monolaurate (Span 20) at a concentration of from 0 mg/mL to about 0.1 mg/mL; (d) sodium dihydrogen phosphate dihydrate at a concentration of from about 0.5 mg/mL to about 1 mg/mL; (e) disodium hydrogen phosphate dihydrate at a concentration of from about 0.5 mg/mL to about 1 mg/mL; and (f) sodium chloride at a concentration of from about 5 mg/mL to about 10 mg/mL. In some embodiments, the liquid pharmaceutical composition comprises: (a) ensifentrine particles at a concentration of 1.2 mg/mL; (b) polysorbate 20 (Tween 20) at a concentration of 0.5 mg/mL; (c) sorbitan monolaurate (Span 20) at a concentration of 0.05 mg/mL; (d) sodium dihydrogen phosphate dihydrate at a concentration of 0.744 mg/mL; (e) disodium hydrogen phosphate dihydrate at a concentration of 0.853 mg/mL; and (f) sodium chloride at a concentration of 8.6 mg/mL. In some embodiments, a total weight of the ensifentrine or the pharmaceutically acceptable salt thereof in the liquid pharmaceutical composition is of from about 2.7 mg to about 3.3 mg. In some embodiments, the ensifentrine or the pharmaceutically acceptable salt thereof is used in combination with a muscarinic receptor antagonist, a beta-adrenergic receptor agonist, or an inhaled corticosteroid. In some embodiments, the COPD is moderate COPD. In some embodiments, the COPD is severe COPD. Provided herein are uses of any of the method provided herein for treating COPD in a human subject.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A shows predicted AUC and FIG. 1B shows predicted Cₘₐₓ ensifentrine blood concentrations (median and 90% prediction interval) in COPD subjects with normal to moderately impaired renal function.

FIG. 2 shows simulated ensifentrine concentration-time profiles at steady state in COPD subjects with normal renal function to mild renal impairment, and with moderate renal impairment.

FIG. 3 shows AUC_{tau,ss} ensifentrine blood concentration for subjects with healthy liver function, moderate hepatic impairment, or severe hepatic impairment.

### DETAILED DESCRIPTION

### Certain Definitions

As used herein and in the appended claims, the singular forms “a,” “and,” and “the” include plural referents unless the context clearly dictates otherwise. Thus, for example, reference to “an agent” includes a plurality of such agents. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “substantially the same” when referring to two numbers or ranges means that the numbers or ranges are within between 1% and 15% of each other. The term “comprising” (and related terms such as “comprise” or “comprises” or “having” or “including”) is not intended to exclude. An embodiment of any composition of matter, composition, method, or process, or the like, described herein, may “consist of” or “consist essentially of” the described features.

As used herein, unless stated otherwise, “%” or “percent” as used herein means percent by weight (e.g., w/w %), percent by volume (e.g., v/v %), molar percentage (e.g., mol/mol %). Preferably, “%” or “percent” as used herein means percent by weight (e.g., w/w %).

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

### Trough Lung Function

The lung function of a COPD subject typically follows a cycle during maintenance therapy. Lung function in COPD can be measured by determining the forced expiratory volume in one second (FEV₁) of a subject. In the period following administration of the drug, improved lung function may be achieved, reaching a peak improvement a certain period after the drug was administered. The lung function of the subject then decreases towards the trough lung function: i.e. the lowest point in the lung function cycle during maintenance therapy. The trough lung function generally occurs shortly before the next administration of the maintenance therapy drug. For instance, for a twice daily (morning and evening) maintenance therapy, trough lung function generally occurs immediately before the morning dose of the drug (morning trough lung function and evening dose of the drug (evening trough lung function).

The trough lung function achieved during a maintenance therapy may have significant effects on the symptoms and/or quality of life of a COPD subject. The trough lung function effectively represents the worst lung function at a stage during the maintenance treatment. For a twice daily dosing, the morning trough lung function will often coincide with a period of time for which the patient is asleep. This means that the patient can have poor lung function for part of the night, disrupting his or her sleep (for instance due to limited oxygen being absorbed) and causing tiredness, thereby reducing quality of life. In some embodiments, this is the case in COPD subjects who already have disturbed sleep, for instance due to sleep disorders and co-morbidities that disrupt sleep.

If trough lung function is improved, symptoms of COPD may be more consistently ameliorated along with quality of life. Improvement in trough lung function may be beneficial in those subjects already susceptible to sleep disruption. While there are a number of drugs disclosed for use in treating COPD, they are not all equally effective specifically in improving trough lung function, and in particular morning trough lung function. It would be clinically advantageous to administer specific drugs which are effective in improving trough lung function to subjects for whom it is a specific therapeutic desire to optimize the therapeutic effect of the pharmacological intervention.

In some embodiments ensifentrine is effective in increasing trough lung functions in subjects with or without COPD. In some instances, ensifentrine is effective in increasing trough lung function in subjects with COPD when used as a maintenance therapy. In some embodiments, ensifentrine may improve morning trough lung function in COPD subjects, which may be beneficial for COPD subjects who are susceptible to sleep disruption, for instance due to the presence of a co-morbidity that affects sleep.

### COPD Exacerbations

In some instances, subjects with COPD also suffer from occasional, temporary worsening of their symptoms (known as a COPD exacerbation). A COPD exacerbation typically involves an increase in breathlessness (dyspnea), the presence of excessive mucus (increased sputum volume), change in mucus color (sputum purulence) and/or an increase in coughing. In some instances, the methods of reducing COPD exacerbations provided herein (e.g., such as by administering ensifentrine) improve dyspnea. In some instances, the methods of reducing COPD exacerbations provided herein (e.g., such as by administering ensifentrine) improve excessive mucus production. In some instances, the methods of reducing COPD exacerbations provided herein (e.g., such as by administering ensifentrine) also decrease frequency of coughing.

In some instances, the methods of reducing COPD exacerbations provided herein (e.g., such as by administering a pharmaceutical composition comprising ensifentrine) decrease the frequency of coughing by at least 10% (e.g., at least 20%, at least 30%, at least 40%, at least 50%, at least 60%, or at least 70%), such as compared to as subject who is not be administered a pharmaceutical composition (e.g., comprising ensifentrine) as provided elsewhere herein. In some instances, the methods of reducing COPD exacerbations provided herein (e.g., such as by administering a pharmaceutical composition comprising ensifentrine) decrease the frequency of coughing by at most 95% (e.g., at most 90%, at most 80%, at most 70%, at most 60%, or at most 50%), such as compared to a subject who is not administered a pharmaceutical composition (e.g., comprising ensifentrine) as provided elsewhere herein. In some embodiments, administration of a pharmaceutical composition described herein (e.g., such as comprising ensifentrine) to a subject reduces COPD exacerbations by at least 20%, at least 30%, at least 40%, at least 50%, at least 60%, or at least 70%.

In some instances, certain patient groups are particularly susceptible to COPD exacerbations. In some instances, this includes subjects suffering from additional respiratory conditions such as asthma and subjects suffering from allergic conditions. Subjects susceptible to COPD exacerbations have typically experienced COPD exacerbations previously. In some embodiments, a patient group particularly susceptible to COPD exacerbations includes subjects with asthma. In some embodiments, a patient group particularly susceptible to COPD exacerbations includes subjects suffering from allergic conditions.

In some instances, COPD exacerbations can be severe and limit the quality of life of subjects with COPD. Not all drugs which are disclosed for use in treating COPD are equally effective in reducing the severity and/or frequency of COPD exacerbations or time to first COPD exacerbation. It may be advantageous for a COPD drug to be used which is effective in reducing the severity and/or frequency of COPD exacerbations when there is a specific clinical need to address such exacerbations.

In some embodiments, ensifentrine is highly effective in reducing the frequency and/or severity of COPD exacerbations in subjects with COPD. In particular, ensifentrine has been found to lead to a more pronounced reduction in COPD exacerbations compared to many known treatments for COPD.

### COPD Severity

The Global Initiative for Chronic Obstructive Lung Disease (GOLD) classifies COPD into four distinct stages: mild COPD, moderate COPD, severe COPD, or very severe COPD. The 2022 COPD report is published by the Global Initiative for Chronic Obstructive Disease, Inc, and that document is incorporated by reference herein in its entirety.

In some embodiments, the abovementioned stages of COPD can be classified as set out below, where FVC is forced vital capacity. In some embodiments, mild COPD is classified as FEV₁/FVC<0.7 and FEV₁≥80% predicted. In some embodiments, moderate COPD is classified as FEV₁/FVC<0.7 and 50%≤FEV₁<80% predicted. In some embodiments, severe COPD is classified as FEV₁/FVC<0.7 and 30%<FEV₁<50% predicted. In some embodiments, very severe COPD is classified as FEV₁/FVC<0.7 and FEV₁<30% predicted.

In some embodiments, the actual FEV₁ for a subject is compared with a predicted FEV₁ value based on factors such as age and height of the subject. These predicted values are readily available to the skilled person, for instance from the National Health and Nutrition Examination Survey III (Hankinson J L, Odencrantz J R, Fedan K B. Spirometry reference values from a sample of the general U.S. Population. Am J Respir Crit Care. 1999; 159:179-187). In some instances, examples of equations for calculating the predicted FEV₁ (in L) for a subject are as follows, where H is height (cm) and A is age (yrs):
Males: 0.0430H−0.0290A−2.490
Females: 0.0395H−0.025A−2.600

The FEV₁ and FVC may be used to determine the severity of COPD in a patient and are measured by carrying out spirometry shortly after administration of an adequate dose of at least one short-acting inhaled bronchodilator. Typically, measurement of FEV₁ and FVC for determining COPD disease severity is done between 15 and 30 minutes following administration of salbutamol (albuterol).

In some embodiments, as used herein, FEV₁ and FVC are determined as set out in the article Standardisation of spirometry, Eur J 2005; 26; 319-338.

In some embodiments, a subject may accordingly be determined to have moderate COPD by measuring FEV₁/FVC<0.7 and 50%≤FEV1<80% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. In some embodiments, a subject may accordingly be determined to have severe COPD by measuring FEV₁/FVC<0.7 and 30%≤FEV1<50% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. The determination of the subject's COPD severity may take place at least 1 day prior to the first administration of the compound (e.g., ensifentrine). The determination of the subject's COPD severity may take place at least 1 week prior to the first administration of the ensifentrine (e.g., or a pharmaceutical composition comprising ensifentrine).

In some instances, as used herein FEV₁ and FVC are determined as set out in the article Standardization of Spirometry, Eur J 2005; 26; 319-338.

In some instances, ensifentrine is effective in treating all forms of COPD. In some instances, ensifentrine is effective in treating moderate COPD. In some instances, ensifentrine is effective in treating mild COPD. In some instances, ensifentrine is effective in treating severe COPD. In some instances, ensifentrine is effective in treating very severe COPD. In some instances, inhaled administration of ensifentrine has been found to result in a significant increase in FEV₁ (forced expiratory volume in 1 second) in subjects with moderate COPD).

In some instances, the pharmaceutical compositions (e.g., comprising ensifentrine) provided herein may provide varying pharmacokinetic (PK) parameters depending on apparent clearance of the ensifentrine. The apparent clearance may be affected by differences in renal impairment or hepatic impairment. Determination of the appropriate blood plasma concentrations (e.g., due to potential modified clearance rates) of ensifentrine to provide to subjects with modified renal or hepatic impairment is desirable to expand the possible patient population to be treated with ensifentrine.

### Renal and Hepatic Function

In some instances, the pharmaceutical compositions (e.g., comprising ensifentrine) provided herein are effective in treating COPD in individuals having varying levels of renal function. In some instances, the pharmaceutical compositions herein are effective in treating COPD in individuals having normal renal function. In some instances, the pharmaceutical compositions herein are effective in treating COPD in individuals having renal impairment. In some embodiments, renal impairment may comprise a mild renal impairment or a moderate renal impairment. In some embodiments, ensifentrine is efficacious in treating COPD in patients with varying levels of renal impairment, the resultant pharmacokinetic (PK) parameters of subjects with normal renal function to mild renal impairment and subjects with moderate renal impairment vary, such as described in Example 1. Renal impairment variations may be associated with reductions in apparent clearance and increases in steady state Cₘₐₓ and AUC (exposure) of ensifentrine.

In some embodiments, the pharmaceutical compositions provided herein are effective in treating COPD in individuals having varying levels of hepatic impairment, such as described in Example 1. In some embodiments, the pharmaceutical compositions are effective in treating COPD in individuals having moderate hepatic impairment. In some embodiments, the pharmaceutical compositions are effective in treating COPD in individuals with severe hepatic impairment. While hepatic function may be a factor in clearance rates (e.g., of ensifentrine) in subjects, it was found that regardless of hepatic impairment, PK parameters, such as steady state AUC, are not meaningfully differentiated from each other, such as described in Example 1. In some instances, the PK parameters of subjects with hepatic impairment are not differentiated from subjects without hepatic impairment.

A “subject” provided herein may also be referred to herein as a “patient”. In any of the embodiments provided herein, a subject may be a human subject. In some embodiments, a subject is a male. In some embodiments, a subject is a female. In some embodiments, a subject has an age of greater than or equal to 65 years. In some embodiments, a subject has an age of less than 65 years. In some embodiments, the subjects provided herein may be classified into one or more subgroup populations. In some embodiments, the subgroup population comprises level of renal impairment. In some embodiments, the subgroup population comprises normal renal function to mild renal impairment. In some embodiments, the subgroup population comprises moderate renal impairment. In some embodiments, the subgroup population comprises a level of hepatic impairment. In some embodiments, the subgroup population comprises normal hepatic function. In some embodiments, the subgroup population comprises moderate hepatic function. In some embodiments, the subgroup population comprises severe hepatic impairment. As described herein, the level of renal or hepatic impairment may modify the clearance rates of administered ensifentrine. As such, desirable blood plasma concentrations for individual subgroups of individuals (e.g., such as those exhibiting modified clearance rates of ensifentrine) are desirable.

In some embodiments, the subjects provided herein may take a background medication. In some embodiments, the background medication is selected from one or more of a long-acting muscarinic antagonist (LAMA), a long-acting beta-agonist (LABA) and an inhaled corticosteroid (ICS). In some embodiments, the background medication is a long-acting muscarinic antagonist (LAMA). In some embodiments, the background medication is a long-acting beta agonist (LABA). In some embodiments, the background medication is an inhaled corticosteroid (ICS). In some embodiments, the subjects provided herein do not take a background medication, which background medication is a long-acting muscarinic antagonist (LAMA), a long-acting beta-agonist (LABA) and an inhaled corticosteroid (ICS).

### Ensifentrine Pharmaceutical Compositions

In some embodiments, provided herein are pharmaceutical compositions comprising ensifentrine or a pharmaceutically acceptable salt thereof. In some embodiments, provided herein are pharmaceutical compositions comprising ensifentrine free base.

In any of the embodiments provided herein, the pharmaceutical compositions may be liquid pharmaceutical compositions.

In some embodiments, the pharmaceutical composition is a suspension formulation (e.g., a suspension of particles comprising the ensifentrine in a diluent). In some embodiments, the pharmaceutical composition is a dry powder. In some embodiments, the dry powder comprises particles comprising the compound and particles of a carrier (e.g., lactose). In some embodiments, the dry powder is dispersed in a diluent, such as a diluent described elsewhere herein.

In some embodiments, the pharmaceutical compositions provided herein are inhalable pharmaceutical compositions. In some embodiments, the inhalable pharmaceutical composition comprises a suspension of particles of ensifentrine in a diluent, such as a diluent described elsewhere herein.

In some embodiments, the pharmaceutical compositions provided herein comprise (e.g., ensifentrine) particles. In some embodiments, the particles comprise ensifentrine or a pharmaceutically acceptable salt. In some embodiments, the particles comprise at least 90% by weight of ensifentrine or a pharmaceutically acceptable salt thereof. In some embodiments, the particles comprise at least 95% by weight of ensifentrine or a pharmaceutically acceptable salt thereof. In some embodiments, the particles comprise at least 97.5% by weight of ensifentrine or a pharmaceutically acceptable salt thereof. In some embodiments, the particles comprise at least 98% by weight of ensifentrine or a pharmaceutically acceptable salt thereof. In some embodiments, the particles comprise at least 99% by weight of ensifentrine or a pharmaceutically acceptable salt thereof. In some embodiments, the particles comprise at least 99.2% by weight of ensifentrine or a pharmaceutically acceptable salt thereof. In some embodiments, the particles consist of ensifentrine or a pharmaceutically acceptable salt thereof. In some embodiments, the particles comprise at least 90% by weight of ensifentrine free base. In some embodiments, the particles comprise at least 95% by weight of ensifentrine free base. In some embodiments, the particles comprise at least 97.5% by weight of ensifentrine free base. In some embodiments, the particles comprise at least 98% by weight of ensifentrine free base. In some embodiments, the particles comprise at least 99% by weight of ensifentrine free base. In some embodiments, the particles comprise at least 99.2% by weight of ensifentrine free base. In some embodiments, the particles consist of ensifentrine free base.

In some embodiments, a particle comprising ensifentrine as used herein may be referred to as an “ensifentrine particle”.

In some embodiments, the ensifentrine provided herein is in crystalline form. In some embodiments, at least 90% of the ensifentrine by weight is in the form of ensifentrine free base Form I. In some embodiments, at least 90% of the particles comprise ensifentrine free base Form I. Ensifentrine free base Form I is a crystalline polymorph of ensifentrine (crystalline polymorph Form I) which typically has a powder X-ray diffraction pattern comprising characteristic peaks at 10.1° and 12.9°±0.1° 2θ. As used herein, values of ° 20 may be measured using an X-ray wavelength of Cu Kα radiation (λ=1.5406 Å). In some embodiments, the powder x-ray diffraction pattern of Form I further comprises characteristic peaks at 15.3° and 17.6°±0.1° 2θ. In some embodiments, Form I of ensifentrine may have a powder X-ray diffraction pattern comprising at least 5 characteristic peaks selected from 6.4°, 10.1°, 12.6°, 12.9°, 13.6°, 14.2°, 14.7°, 15.3°, 15.4°, 15.8°, 17.0°, 17.6°, 18.9°, 20.9°, 22.4°, 22.8° and 28.7±0.1° 2θ. In some embodiments, crystalline polymorph Form I has a differential scanning calorimetry trace showing a maximum at 248° C.

In some embodiments, the powder x-ray diffraction pattern of Form I of ensifentrine comprises at least 5 (e.g., at least 3, 4, 6, 7, or 8) characteristic peaks, in terms of 2θ, selected from about 6.4°, about 10.1°, about 12.6°, about 12.9°, about 13.6°, about 14.2°, about 14.7°, about 15.3°, about 15.4°, about 15.8°, about 17.0°, about 17.6°, about 18.9°, about 20.9°, about 22.4°, about 22.8°, and about 28.7°.

In some embodiments, the particles comprise at least 98% by weight of ensifentrine crystalline polymorph Form I. In some embodiments, the (e.g., ensifentrine) particles comprise at least 99% by weight of ensifentrine crystalline polymorph Form I.

In some embodiments, ensifentrine crystalline polymorph Form I has the following structural parameters obtained by single crystal analysis:

| Wavelength | 0.71073 Å | |
|---|---|---|
| Crystal System | Triclinic | |
| Space Group | P-1 | |
| Unit Cell Dimensions | a = 8.1246(4) Å | α = 91.583(4)° |
| b | b = 11.4573(5) Å | β = 90.299(4)° |
| c | c = 13.2398(6) Å | γ = 99.628(4)° |
| Volume | 1214.56(10) Å 3 | |
| C | 2. | |

In some instances, the crystalline polymorph Form I is the most thermodynamically stable polymorphic form of ensifentrine and is also expected to have the longest shelf life storage. It is expected that this advantage should extend further to the potential shelf life of any commercial ensifentrine drug product comprising the stable crystalline form I as an API. Furthermore, crystalline polymorph I is more amenable for the development of a reproducible uniform micronized dry solid powder of ensifentrine for certain pharmaceutical formulation applications, such as preparation of formulations described herein.

### Impurity Profile

It has been found that known processes for producing ensifentrine are associated with undesirable levels of impurities. For example, the process described in WO 00/58308 A1 in which a urea group is added using sodium cyanate and aqueous hydrochloric acid produces and ensifentrine substance containing a biuret impurity which could not be readily removed. WO 2018/020249 A1 discusses several possible reagents which may be used to add a urea group in the final step of the production of ensifentrine, but no detailed discussion of the conditions of the final ureation step is provided. It may be desirable to utilize synthetic processes for the production of ensifentrine which produce a drug substance with a favorable impurity profile without the need for extensive purification and recrystallization, and in particular, where the drug substance comprises low levels of a biuret impurity. In some instances, it has been found that it is possible to produce ensifentrine with a favorable impurity profile, and in particular ensifentrine having low levels of a biuret impurity, by reaction comprising reacting an amine intermediate with 4-nitrophenyl chloroformate and ammonia in a solvent comprising dichloromethane.

In some embodiments, the (e.g., ensifentrine) particles provided herein comprise 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU). In some embodiments, the structure of BMIQU is:

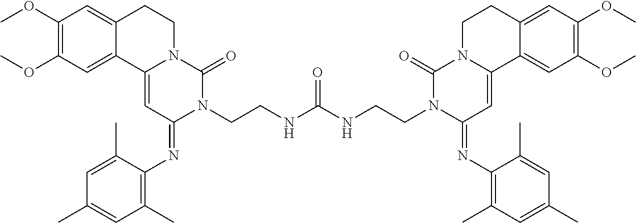

In some embodiments, the (e.g., ensifentrine) particles provided herein comprise the BMIQU in an amount of from 0% to about 0.6% by weight. In some embodiments, the particles comprise BMIQU in an amount of at least about 0.005% by weight. In some embodiments, the particles comprise BMIQU in an amount of at least about 0.01% by weight. In some embodiments, the particles comprise BMIQU in an amount of at least about 0.1% by weight. In some embodiments, the particles comprise BMIQU in an amount of at most about 1% by weight. In some embodiments, the particles comprise BMIQU in an amount of at most about 0.6% by weight. In some embodiments, the particles comprise BMIQU in an amount of at most about 0.3% by weight. In some embodiments, the particles comprise BMIQU in an amount of from about 0.01% to about 0.3% by weight. In some embodiments, the particles comprise BMIQU in an amount of from about 0.3% to about 0.6% by weight. In some embodiments, the particles comprise BMIQU in an amount of from about 0.02% to about 0.06% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles provided herein comprise a biuret impurity of formula (A):

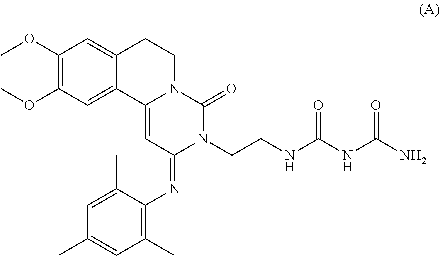 [SMILES: COc1cc2c(cc1OC)-c1c/c(=N\c3c(C)cc(C)cc3C)n(CCNC(=O)NC(N)=O)c(=O)n1CC2]

In some embodiments, the (e.g., ensifentrine) particles comprise the biuret impurity of formula (A) in an amount of from 0% to about 0.5% by weight. In some embodiments, the particles comprise the biuret impurity of formula (A) in an amount of at least 0.01% by weight. In some embodiments, the particles comprise the biuret impurity of formula (A) in an amount of at least 0.05% by weight. In some embodiments, the particles comprise the biuret impurity of formula (A) in an amount of at least 0.1% by weight. In some embodiments, the particles comprise the biuret impurity of formula (A) in an amount of at least 0.3% by weight. In some embodiments, the particles comprise the biuret impurity of formula (A) in an amount of at most 0.7% by weight. In some embodiments, the particles comprise the biuret impurity of formula (A) in an amount of at most 0.5% by weight. In some embodiments, the particles comprise the biuret impurity of formula (A) in an amount of at most 0.3% by weight. In some embodiments, the particles comprise the biuret impurity of formula (A) in an amount of at most 0.2% by weight. In some embodiments, the particles comprise the biuret impurity of formula (A) in an amount of from 0% to about 0.3% by weight. In some embodiments, the particles comprise the biuret impurity of formula (A) in an amount of from 0% to about 0.05% by weight. In some embodiments, the particles comprise the biuret impurity of formula (A) in an amount of from 0% to about 0.03% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise 1-(2-(9-hydroxy-2-(mesitylimino)-10-methoxy-4-oxo-6,7-dihydro-2H-pyrimido[6,1-a]isoquinolin-3(4H)-yl)ethyl)urea (9-des-methyl impurity). In some embodiments, the (e.g., ensifentrine) particles comprise 1-(2-(10-hydroxy-2-(mesitylimino)-9-methoxy-4-oxo-6,7-dihydro-2H-pyrimido[6,1-a]isoquinolin-3(4H)-yl)ethyl)urea (10-des-methyl impurity). In some embodiments, the particles comprise the 9-des-methyl impurity and the 10-des-methyl impurity. In some embodiments, the particles comprise the 9-des-methyl or the 10-des-methyl impurity.

In some embodiments, the 9-des-methyl impurity has the structure:

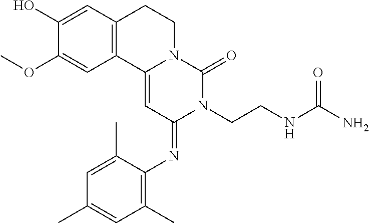 [SMILES: COc1cc2c(cc1O)CCn1c-2c/c(=N\c2c(C)cc(C)cc2C)n(CCNC(N)=O)c1=O]

In some embodiments, the 10-des-methyl impurity as the structure:

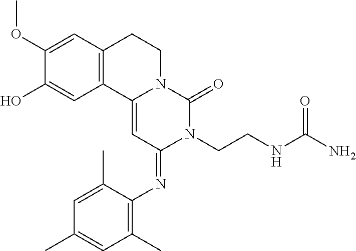 [SMILES: COc1cc2c(cc1O)-c1c/c(=N\c3c(C)cc(C)cc3C)n(CCNC(N)=O)c(=O)n1CC2]

In some embodiments, the (e.g., ensifentrine) particles comprise the 9-des-methyl impurity in an amount of from 0% to about 0.1% by weight. In some embodiments, the particles comprise the 9-des-methyl impurity in an amount of at least 0.01% by weight. In some embodiments, the particles comprise the 9-des-methyl impurity in an amount of at least 0.05% by weight. In some embodiments, the particles comprise the 9-des-methyl impurity in an amount of at least 0.1% by weight. In some embodiments, the particles comprise the 9-des-methyl impurity in an amount of at most 0.2% by weight. In some embodiments, the particles comprise the 9-des-methyl impurity in an amount of 0.1% by weight. In some embodiments, the particles comprise the 9-des-methyl impurity in an amount of from about 0.01% to about 0.1% by weight. In some embodiments, the particles comprise the 9-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise the 10-des-methyl impurity in an amount of from 0% to about 0.1% by weight. In some embodiments, the particles comprise the 10-des-methyl impurity in an amount of at least 0.01% by weight. In some embodiments, the particles comprise the 10-des-methyl impurity in an amount of at least 0.05% by weight. In some embodiments, the particles comprise the 10-des-methyl impurity in an amount of at least 0.1% by weight. In some embodiments, the particles comprise the 10-des-methyl impurity in an amount of at most 0.2% by weight. In some embodiments, the particles comprise the 10-des-methyl impurity in an amount of 0.1% by weight. In some embodiments, the particles comprise the 10-des-methyl impurity in an amount of from about 0.01% to about 0.1% by weight. In some embodiments, the particles comprise the 10-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise (E)-2-(mesitylimino)-9,10-dimethoxy-2,3,6,7-tetrahydro-4H-pyrimido[6,1-a]isoquinolin-4-one (compound (I)). In some embodiments, the (e.g., ensifentrine) particles comprise (E)-3-(2-aminoethyl)-2-(mesitylimino)-9,10-dimethoxy-2,3,6,7-tetrahydro-4H-pyrimido[6,1-a]isoquinolin-4-one (compound (IV)). In some embodiments, the particles comprise both compound (I) and compound (IV). In some embodiments, the particles comprise compound (I) or compound (IV).

In some embodiments, the structure of compound (I) is:

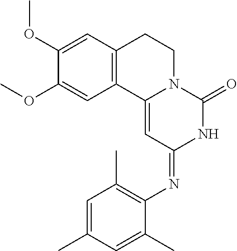 [SMILES: COc1cc2c(cc1OC)-c1c/c(=N\c3c(C)cc(C)cc3C)[nH]c(=O)n1CC2]

In some embodiments, the structure of compound (IV) is:

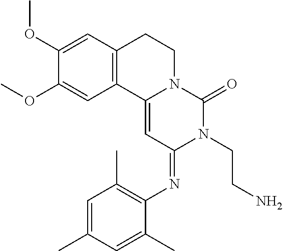 [SMILES: COc1cc2c(cc1OC)-c1c/c(=N\c3c(C)cc(C)cc3C)n(CCN)c(=O)n1CC2]

In some embodiments, the (e.g., ensifentrine) particles comprise from 0% to about 0.04% by weight of compound (I). In some embodiments, the particles comprise at least 0.01% by weight of compound (I). In some embodiments, the particles comprise at least 0.02% by weight of compound (I). In some embodiments, the particles comprise at most 0.05% by weight of compound (I). In some embodiments, the particles comprise at most 0.04% by weight of compound (I). In some embodiments, the (e.g., ensifentrine) particles comprise from 0% to about 0.04% by weight of compound (IV). In some embodiments, the particles comprise at least 0.01% by weight of compound (IV). In some embodiments, the particles comprise at least 0.02% by weight of compound (IV). In some embodiments, the particles comprise at most 0.05% by weight of compound (IV). In some embodiments, the particles comprise at most 0.04% by weight of compound (IV). In some embodiments, the particles do not comprise compound (I). In some embodiments, the particles do not comprise compound (IV). In some embodiments, the particles do not comprise compound (I) or compound (IV). In some embodiments, the (e.g., ensifentrine) particles comprise from 0% to about 0.04% by weight total of compound (I) and compound (IV). In some embodiments, the particles comprise at least 0.01% by weight total of compound (I) and compound (IV). In some embodiments, the particles comprise at least 0.02% by weight total of compound (I) and compound (IV). In some embodiments, the particles comprise at most 0.05% by weight total of compound (I) and compound (IV). In some embodiments, the particles comprise at most 0.04% by weight total of compound (I) and compound (IV). In some embodiments, the weight percentage is relative to the total weight of ensifentrine (e.g., free base). In some embodiments, the weight percentage is relative to the total weight of the particles.

In some embodiments, the BMIQU, biuret impurity, 9-des-methyl impurity, 10-des-methyl impurity, compound (I), and compound (IV), if present, may optionally be present in the form of a salt. In certain embodiments, the BMIQU, biuret impurity, 9-des-methyl impurity, 10-des-methyl impurity, compound (I), and compound (IV) are present in free base form.

In some embodiments, the (e.g., ensifentrine) particles provided herein comprise ensifentrine (e.g., ensifentrine free base (e.g., in crystalline Form I)), BMIQU, biuret impurity, 9-des-methyl impurity, and 10-des-methyl impurity. In some embodiments, the (e.g., ensifentrine) particles provided herein consists of ensifentrine (e.g., ensifentrine free base (e.g., in crystalline Form I)), BMIQU, biuret impurity, 9-des-methyl impurity, and 10-des-methyl impurity.

In some embodiments, the (e.g., ensifentrine) particles provided herein comprise from about 99.4% to about 99.9% by weight of ensifentrine, from about 0.01% to about 0.3% by weight of BMIQU, from 0% to about 0.1% by weight of the biuret impurity, from about 0.01% to about 0.2% by weight of the 9-des-methyl impurity, and from about 0.01% to about 0.2% by weight of the 10-des-methyl impurity, wherein the weight percent is relative to the total weight of the particles.

In some embodiments, the (e.g., ensifentrine) particles provided herein comprise from about 99.5% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

In some embodiments, the (e.g., ensifentrine) particles provided herein consists of from about 99.6% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

In some embodiments, provided herein are particles comprising ensifentrine (e.g., ensifentrine free base). In some embodiments, the particles have a particle size distribution with a Dv50 of from about 0.5 μm to about 5.0 μm. In some embodiments, the particles comprise a Dv50 of from about 1.0 μm to about 2.0 μm. In some embodiments, the particles comprise a Dv90 of from about 1.8 μm to about 2 μm. In some embodiments, the particles comprise a Dv90 of from about 2.5 μm to about 6.0 μm. In some embodiments, the particles comprise a Dv90 of from about 2.8 μm to about 4.2 μm. In some embodiments, the particles comprise a Dv10 of from about 0.2 μm to about 1.0 μm In some embodiments, the particles comprise a Dv10 of from about 0.4 μm to about 0.6 μm. In some embodiments, the particles comprise a Dv10 of from about 0.4 μm to about 0.6 μm and a Dv90 of from about 2.8 μm to about 4.2 μm. In certain embodiments, the particles comprise a Dv90 that is not more than 4.5 μm. In some embodiments, the particles comprise a Dv90 of about 2, 2.1, 2.2, 2.3, 2.5, 2.5, or 2.6 μm.

In some embodiments, particle sizes are described herein by reference to the Dv50 value, which is the median particle size for a volume distribution. Thus, half of the volume of the particles have diameters of less than the Dv50 value and half the volume of the particles have diameters of greater than the Dv50 value. Similarly, in some embodiments, particle sizes are described herein by their Dv90 or Dv10 values. The skilled artisan would understand this to be a well-known parameter in which to describe particle size distributions.

The technique used to measure particle size (e.g., Dv10, Dv50, Dv90 values) as described herein may be laser diffraction.
For instance, the particle size distribution can be measured by laser diffraction using a Malvern Mastersizer 3000. Typically, the instrument parameters for the Malvern Mastersizer 3000 are as follows:
Non-spherical particle mode: Yes
Refractive index: 1.500
Absorption index: 0.500
Particle density: 1.00 g/cm³
Dispersant name: 0.02% Polysorbate 20 in water
Refractive index: 1.330
Stirrer; 1000 rpm
Sample measurement: 10 s
Obscuration: 10-20%

The particles described herein may comprise ensifentrine (e.g., ensifentrine free base). In some embodiments, the particles comprise at least 90 wt % ensifentrine free base relative to the total weight of the particles. In some embodiments, the particles comprise at least 95 wt % ensifentrine free base. In some embodiments, the particles comprise at least 97 wt % ensifentrine free base. In some embodiments, the particles comprise at least 99 wt % ensifentrine free base. In some embodiments, the particles comprise at least 99.5 wt % ensifentrine free base. In some instances, the particles consist of ensifentrine.

The (e.g., ensifentrine) particles comprising ensifentrine (e.g., ensifentrine free base) may be present in the pharmaceutical compositions in any suitable concentration according to one of skill in the art. In some embodiments, particles comprising ensifentrine are present in the pharmaceutical compositions at a concentration of about 0.1 mg/mL to about 5.0 mg/mL. In some embodiments, the particles are present in the pharmaceutical compositions at a concentration of about 0.1 mg/mL to about 2.5 mg/mL. In some embodiments, the particles are present in the pharmaceutical compositions at a concentration of about 1.0 mg/mL to about 2.0 mg/mL. In some embodiments, the particles comprising ensifentrine are present in the pharmaceutical compositions at a concentration of (e.g., about) 1.2 mg/mL.

In some embodiments, the concentration of ensifentrine or a pharmaceutically acceptable salt thereof (e.g., ensifentrine free base) in the pharmaceutical composition is at least 0.8 mg/mL. In some embodiments, the concentration of ensifentrine or a pharmaceutically acceptable salt thereof (e.g., ensifentrine free base) is at least about 0.9 mg/mL (e.g., 1 mg/mL, 1.1 mg/mL). In some embodiments, the concentration of ensifentrine or a pharmaceutically acceptable salt thereof (e.g., ensifentrine free base) is at most 1.5 mg/mL. the concentration of ensifentrine or a pharmaceutically acceptable salt thereof (e.g., ensifentrine free base) is at most 1.4 mg/mL (e.g., 1.3 mg/mL). In some embodiments, the concentration of ensifentrine or a pharmaceutically acceptable salt thereof (e.g., ensifentrine free base) is from about 1 mg/mL to about 1.4 mg/mL (e.g., 1.1 mg/mL to 1.3 mg/mL). In certain embodiments, the concentration of ensifentrine or a pharmaceutically acceptable salt thereof (e.g., ensifentrine free base) is about 1.2 mg/mL. In some embodiments, the concentration of ensifentrine free base in the pharmaceutical composition is (e.g., about) 1.2 mg/mL.

In some embodiments, the pharmaceutical compositions provided herein further comprise one or more tonicity adjusters, one or more buffers, and one or more surfactants. In some embodiments, the pharmaceutical composition further comprises one or more tonicity adjusters. In some embodiments, the pharmaceutical composition further comprises one or more buffers. In some embodiments, the pharmaceutical composition further comprises one or more surfactants.

In some embodiments, the pharmaceutical compositions provided herein further comprise one or more tonicity adjusters. In some embodiments, the tonicity adjuster comprises sodium chloride. In some embodiments, the tonicity adjuster (e.g., sodium chloride) is present in the pharmaceutical composition at a concentration of at least 1 mg/mL (e.g., 2 mg/mL, 3 mg/mL, 4 mg/mL, 5 mg/mL, 6 mg/mL, 7 mg/mL). In some embodiments, the tonicity adjuster (e.g., sodium chloride) is present in the pharmaceutical composition at a concentration of at most 15 mg/mL (e.g., 14 mg/mL, 13 mg/mL, 12 mg/mL, 11 mg/mL, 10 mg/mL, 9 mg/mL). In some embodiments, the tonicity adjuster (e.g., sodium chloride) is present in the pharmaceutical composition at a concentration of from about 1 mg/mL to about 15 mg/mL, about 3 mg/mL to about 10 mg/mL, about 4 mg/mL to about 10 mg/mL, about 5 mg/mL to about 11 mg/mL, about 5 mg/mL to about 9 mg/mL, or about 6 mg/mL to about 9 mg/mL. In some embodiments, the tonicity adjuster (e.g., sodium chloride) is present in the pharmaceutical composition at a concentration of about 7 mg/mL to about 10 mg/mL. In some embodiments, the tonicity adjuster (e.g., sodium chloride) is present in the pharmaceutical composition at a concentration of 8 mg/mL to 9 mg/mL. In certain embodiments, the tonicity adjuster (e.g., sodium chloride) is present in the pharmaceutical composition at a concentration of (e.g., about) 8.6 mg/mL.

In some embodiments, the pharmaceutical compositions provided herein further comprise one or more buffers. In some embodiments, the one or more buffers comprise one or more of a citrate buffer, a phosphate buffer, an acetate buffer, and/or a bicarbonate buffer. In certain embodiments, the buffer is a phosphate buffer. In some embodiments, the buffer comprises sodium dihydrogen phosphate dihydrate and/or disodium hydrogen phosphate dihydrate. In some embodiments, the buffer (e.g., sodium dihydrogen phosphate dihydrate and/or disodium hydrogen phosphate dihydrate) is present in the pharmaceutical composition at a concentration of at least 0.1 mg/mL (e.g., 0.2 mg/mL, 0.4 mg/mL, 0.8 mg/mL, 1 mg/mL, or 1.4 mg/mL). In some embodiments, the buffer (e.g., sodium dihydrogen phosphate dihydrate and/or disodium hydrogen phosphate dihydrate) is present in the pharmaceutical composition at a concentration of 4 mg/mL (e.g., 3.8 mg/mL, 3.3 mg/mL, 2.8 mg/mL, 2.5 mg/mL, or 2 mg/mL). In some embodiments, the buffer (e.g., sodium dihydrogen phosphate dihydrate and/or disodium hydrogen phosphate dihydrate) is present in the pharmaceutical composition at a concentration of about 1 mg/mL to about 2 mg/mL, about 1.2 mg/mL to about 1.8 mg/mL, or about 1.4 mg/mL to about 1.7 mg/mL. In some embodiments, the buffer (e.g., sodium dihydrogen phosphate dihydrate and/or disodium hydrogen phosphate dihydrate) is present in the pharmaceutical composition at a concentration of about 1.5 mg/mL to about 1.7 mg/mL.

In some embodiments, the pharmaceutical composition comprises sodium dihydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL). In some embodiments, the pharmaceutical composition comprises sodium dihydrogen phosphate dihydrate at a concentration of at most 1.2 mg/mL (e.g., 1.1 mg/mL, 1.0 mg/mL, 0.9 mg/mL, 0.8 mg/mL). In some embodiments, the pharmaceutical composition comprises sodium dihydrogen phosphate dihydrate at a concentration of from about 0.5 mg/mL to about 0.9 mg/mL. In some embodiments, the pharmaceutical composition comprises sodium dihydrogen phosphate dihydrate at a concentration of about 0.7 mg/mL to about 0.8 mg/mL. In some embodiments, the pharmaceutical composition comprises sodium dihydrogen phosphate dihydrate at a concentration of about 0.7 mg/mL. In certain embodiments, the pharmaceutical composition comprises sodium dihydrogen phosphate dihydrate at a concentration of (e.g., about) 0.744 mg/mL.

In some embodiments, the pharmaceutical composition comprises disodium hydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL, 0.7 mg/mL). In some embodiments, the pharmaceutical composition comprises disodium hydrogen phosphate dihydrate at a concentration of at most 1.2 mg/mL (e.g., 1.1 mg/mL, 1.0 mg/mL, 0.9 mg/mL). In some embodiments, the pharmaceutical composition comprises disodium hydrogen phosphate dihydrate at a concentration of from about 0.7 mg/mL to about 1 mg/mL. In some embodiments, the pharmaceutical composition comprises disodium hydrogen phosphate dihydrate at a concentration of about 0.8 mg/mL to about 0.9 mg/mL. In some embodiments, the pharmaceutical composition comprises disodium hydrogen phosphate dihydrate at a concentration of about 0.8 mg/mL. In some embodiments, the pharmaceutical composition comprises disodium hydrogen phosphate dihydrate at a concentration of about 0.9 mg/mL. In certain embodiments, the pharmaceutical composition comprises disodium hydrogen phosphate dihydrate at a concentration of (e.g., about) 0.853 mg/mL.

In some embodiments, the pharmaceutical compositions provided herein further comprise one or more surfactants. In some embodiments, the one or more surfactants comprise one or more of lecithin, oleic acid, polyoxyethylene glycol alkyl ethers (e.g., PEG 300, PEG 600, PEG 1000, Brij 30, Brij 35, Brij 56, Brij 76 and Brij 97), polypropylene glycol (e.g., PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (e.g., polysorbates, for instance polysorbate 20, polysorbate 40, polysorbate 60 and polysorbate 80), sorbitan alkyl esters (e.g., sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (e.g., Pluronic surfactants), polyvinyl pyrrolidone K25, polyvinyl alcohol, oligolactic acid, sodium dioctyl sulfosuccinate and polyethoxylated tallow amine (POEA). In certain embodiments, the one or more embodiments, comprise a polysorbate and/or a sorbitan alkyl ester. In some embodiments, the one or more surfactants comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). In some embodiments, the one or more surfactants comprise sorbitan monolaurate (Span 20), sorbitan monooleate (Span 80) or sorbitan trioleate (Span 85). In some embodiments, the pharmaceutical composition comprises polysorbate 20 (Tween 20) and/or sorbitan monolaurate (Span 20). In some instances, the surfactants act as wetting agents in the (e.g., liquid) pharmaceutical compositions described herein.

In some embodiments, the pharmaceutical composition comprises the one or more surfactants (e.g., polysorbate 20 and/or sorbitan monolaurate) in a concentration of at least 0.01 mg/mL (e.g., 0.05 mg/mL, 0.1 mg/mL, 0.2 mg/mL, 0.3 mg/mL, 0.4 mg/mL). In some embodiments, the pharmaceutical composition comprises the one or more surfactants (e.g., polysorbate 20 and/or sorbitan monolaurate) in a concentration of at most 1 mg/mL (e.g., 0.9 mg/mL, 0.8 mg/mL, 0.7 mg/mL, 0.6 mg/mL). In some embodiments, the pharmaceutical composition comprises the one or more surfactants (e.g., polysorbate 20 and/or sorbitan monolaurate) in a concentration of from about 0.3 mg/mL to about 0.7 mg/mL. In some embodiments, the pharmaceutical composition comprises the one or more surfactants (e.g., polysorbate 20 and/or sorbitan monolaurate) at a concentration of from about 0.01 mg/mL to 2 mg/mL. In some embodiments, the pharmaceutical composition comprises the one or more surfactants (e.g., polysorbate 20 and/or sorbitan monolaurate) in a concentration of from about 0.4 mg/mL to about 0.6 mg/mL. In some embodiments, the pharmaceutical composition comprises the one or more surfactants (e.g., polysorbate 20 and/or sorbitan monolaurate) at a concentration of about 0.5 mg/mL. In some embodiments, the pharmaceutical composition comprises the one or more surfactants (e.g., polysorbate 20 and/or sorbitan monolaurate) at a concentration of about 0.6 mg/mL. In certain embodiments, the pharmaceutical composition comprises the one or more surfactants (e.g., polysorbate 20 and/or sorbitan monolaurate) in a concentration of (e.g., about) 0.55 mg/mL.

In some embodiments, the pharmaceutical composition comprises polysorbate 20 (e.g., Tween 20) in a concentration of at least 0.1 mg/mL (e.g., 0.2 mg/mL, 0.3 mg/mL, 0.4 mg/mL). In some embodiments, the pharmaceutical composition comprises polysorbate 20 (e.g., Tween 20) in a concentration of at most 1 mg/mL (e.g., 0.9 mg/mL, 0.8 mg/mL, 0.7 mg/mL, 0.6 mg/mL). In some embodiments, the pharmaceutical composition comprises polysorbate 20 (e.g., Tween 20) in a concentration of from about 0.3 mg/mL to about 0.7 mg/mL. In some embodiments, the pharmaceutical composition comprises polysorbate 20 (e.g., Tween 20) in a concentration of about 0.4 mg/mL to about 0.6 mg/mL. In certain embodiments, the pharmaceutical composition comprises polysorbate 20 (e.g., Tween 20) in a concentration of about 0.5 mg/mL.

In some embodiments, the pharmaceutical composition comprises sorbitan monolaurate (Span 20) in a concentration of at least 0.01 mg/mL (e.g., 0.02 mg/mL, 0.03 mg/mL, 0.04 mg/mL). In some embodiments, the pharmaceutical composition comprises sorbitan monolaurate (Span 20) in a concentration of at most 0.1 mg/mL (e.g., 0.09 mg/mL, 0.08 mg/mL, 0.07 mg/mL, 0.06 mg/mL). In some embodiments, the pharmaceutical composition comprises sorbitan monolaurate (Span 20) in a concentration of from about 0.03 mg/mL to about 0.07 mg/mL. In some embodiments, the pharmaceutical composition comprises sorbitan monolaurate (Span 20) in a concentration of from about 0.04 mg/mL to about 0.06 mg/mL. In certain embodiments, the pharmaceutical composition comprises sorbitan monolaurate (Span 20) in a concentration of (e.g., about) 0.05 mg/mL.

In some embodiments, the pharmaceutical compositions provided herein comprise:
a. water;
b. particles consisting of ensifentrine free base at a concentration of from about 0.1 mg/mL to about 20 mg/mL;
c. one or more tonicity adjusters at a concentration of from about 1.0 mg/mL to about 15 mg/mL;
d. one or more buffers at a total concentration of from about 0.1 mg/mL to about 4.0 mg/mL; and
e. one or more surfactants at total concentration of from about 0.05 mg/mL to about 3 mg/mL.

In some embodiments, the liquid pharmaceutical compositions provided herein comprise:
a. water;
b. particles consisting of ensifentrine free base at a concentration of from about 0.5 mg/mL to about 6 mg/mL;
c. sodium chloride at a concentration of from about 5 mg/mL to about 12 mg/mL;
d. sodium dihydrogen phosphate dihydrate at a concentration of from about 0.3 mg/mL to about 2 mg/mL;
e. disodium hydrogen phosphate dihydrate at a concentration of from about 0.3 mg/mL to about 2 mg/mL; and
f. sorbitan monolaurate at a concentration of from about 0.01 mg/mL to about 0.5 mg/mL.

In some embodiments, the pharmaceutical compositions herein are provided in an ampule, such as for administration by inhalation using a nebulizer. In some embodiments, the ampule comprises the suspension of ensifentrine particles as described herein. In some embodiments, the ampule comprises about 2.6 mL of the pharmaceutical composition. In some embodiments, the ampule comprises at least 3 mg of ensifentrine (e.g., ensifentrine free base). In some embodiments, the ampule comprises about 3.1 mg of ensifentrine. In some embodiments, the ampule is a low density polyethylene (LDPE) ampule.

In some instances, the pharmaceutical compositions provided herein may be referred to as ensifentrine compositions.

In some embodiments, ensifentrine or a pharmaceutically acceptable salt as described herein may be used in combination with a second active agent. In some embodiments, the ensifentrine or pharmaceutically salt thereof may be administered separately or simultaneously with the second active agent. In some embodiments, the subject may already be taking a second active agent as a background therapy for COPD. Alternatively, treatment with the second active agent may start at around the same time as treatment with ensifentrine. The ensifentrine and the second active agent may be administered in a fixed combination.

In some embodiments, the second active agent as provide herein is a muscarinic receptor antagonist, a beta adrenergic receptor agonist, or an inhaled corticosteroid. In some embodiments, the ensifentrine or pharmaceutically acceptable salt may be used in combination with muscarinic receptor antagonist, a beta adrenergic receptor agonist, or an inhaled corticosteroid. The second active agent may be a long-acting muscarinic receptor antagonist (LAMA) or a long-acting beta-adrenergic receptor agonist (LABA). In some embodiments, the second active agent is a muscarinic receptor antagonist. In some embodiments, the second active agent is a beta adrenergic receptor agonist. In some embodiments, the second active agent is an inhaled corticosteroid.

In some embodiments, non-limiting examples of LAMAs include aclidinium, darotropium, tiotropium, glycopyrrolate and umeclidinium. In some embodiments, non-limiting examples of LABAs include salmeterol, formoterol, indacaterol, vilanterol, olodaterol, abediterol and carmoterol. In some embodiments, non-limiting examples of inhaled corticosteroids include beclomethosone, budesonide, fluticasone propionate, ciclesonide, mometasone and fluticasone furoate.

In some embodiments, the subjects provided herein may be using a beta-agonist, such as salbutamol, as a rescue medication.

### Methods of Treating COPD

Provided herein are methods of treating chronic obstructive pulmonary disease (COPD) (e.g., of varying severity), such as COPD provided elsewhere herein. In some embodiments, the methods provided herein comprise administering to a subject in need thereof an (e.g., liquid) pharmaceutical composition, such as a pharmaceutical composition provided elsewhere herein, comprising a therapeutically effective amount of ensifentrine. In some embodiments, the methods provided herein comprise administering to the subject in need thereof (e.g., a therapeutically effective amount of) ensifentrine.

In some embodiments, the methods provided herein are effective in treating mild COPD, moderate COPD, severe COPD, or very severe COPD. In some embodiments, the methods provided herein are effective in treating mild COPD. In some embodiments, the methods provided herein are effective in treating moderate COPD. In some embodiments, the methods provided herein are effective in treating severe COPD. In some embodiments, the methods provided herein are effective in treating very severe COPD. In some embodiments, the methods provided herein are effective in treating moderate or severe COPD, such as moderate or severe COPD such as classified as described elsewhere herein.

### COPD Subgroup Populations

In some embodiments, provided herein are methods of treating COPD in specific subgroup populations who may exhibit varying clearance rates of the administered ensifentrine.

Cₘₐₓ, AUC₀₋ₜₐᵤ and Tₘₐₓ are pharmacokinetic parameters well known to the skilled person. Cₘₐₓ is the maximum concentration of the compound in the blood plasma achieved following administration of the compound. Tₘₐₓ is the time at which Cₘₐₓ observed. AUC is the area under the curve of the blood plasma concentration of the compound as a function of time following administration over a given period. AUC₀₋ₜₐᵤ is the AUC from administration to the end of the dosing period (which may for instance be 8 h, 12 h or 24 h). As used herein, tau is typically 12 h and AUC₀₋ₜₐᵤ is AUC₀₋₁₂ₕ. In some instances, tau is 4 h.

In some embodiments, ensifentrine is effective in increasing lung function (e.g., FEV₁) in subjects with mild or moderate COPD. In some embodiments, ensifentrine is effective in increasing lung function in subjects with severe or very severe COPD. In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of at least 0.08 L (80 mL). In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of at least 0.09 L (e.g., 0.1 L, 0.11 L). In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of at most 0.14 L (140 mL). In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of at most 0.13 L (e.g., 0.12 L, 0.11 L). In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of about 0.08 L to about 0.14 L. In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of about 0.09 L to about 0.13 L. In some embodiments, administration of a pharmaceutical composition (e.g., comprising ensifentrine) to a subject having mild or moderate COPD provides a FEV₁ AUC₀₋₁₂ₕ (e.g., after 12 weeks of administration) of about 0.11 L (110 mL).

In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of at least 0.03 L (30 mL). In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of 0.04 L (e.g., 0.05 L, 0.06 L). In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of at most 0.09 L (e.g., 0.08 L, 0.07 L, 0.06 L). In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of about 0.03 L to about 0.09 L. In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of about 0.05 L to about 0.07 L. In some embodiments, administration of a pharmaceutical composition (e.g., comprising ensifentrine) to a subject having severe or very severe COPD provides a FEV₁ AUC₀₋₁₂ₕ (e.g., after 12 weeks of administration) of about 0.06 L (60 mL).

In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at least 0.14 L (e.g., 140 mL). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at least 0.15 L (e.g., 0.16 L, 0.17 L). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at most 0.2 L (e.g., 0.19 L, 0.18 L, 0.17 L). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.14 L to about 0.2 L. In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.15 L to about 0.18 L. In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.17 L (170 mL).

In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at least 0.08 L (80 mL). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at least 0.09 L (e.g., 0.1 L, 0.11 L). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at most 0.15 L (150 mL). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at most 0.15 L (e.g., 0.14 L, 0.13 L, or 0.12 L). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of from about 0.08 L to about 0.15 L. In some embodiments, administration of a pharmaceutical composition (e.g., comprising ensifentrine) to a subject having severe or very severe COPD provides a change from baseline FEV₁ to peak FEV₁ (e.g., after 12 weeks of administration) of from about 0.1 L to about 0.14 L. In some embodiments, administration of a pharmaceutical composition (e.g., comprising ensifentrine) to a subject having severe or very severe COPD provides a change from baseline FEV₁ to peak FEV₁ (e.g., after 12 weeks of administration) of about 0.11 L.

In some embodiments, the change in baseline FEV₁ to peak FEV₁ for a subject having mild or moderate COPD is at least 30% greater than for a subject having severe or very severe COPD. In some embodiments, the change in baseline to peak FEV₁ for a subject having mild or moderate COPD is at least 40% greater than for a subject having severe or very severe COPD. In some embodiments, the change in baseline to peak FEV₁ for a subject having mild or moderate COPD is from about 40% to about 60% greater than for a subject having severe of very severe COPD. In some embodiments, the change in baseline to peak FEV₁ for a subject having mild or moderate COPD is about 55% greater than for a subject having severe or very severe COPD.

In some embodiments, the change in baseline FEV₁ to peak FEV₁ for a subject having severe or very severe COPD is at least 30% greater than for a subject having mild or moderate COPD. In some embodiments, the change in baseline to peak FEV₁ for a subject having severe or very severe COPD is at least 40% greater than for a subject having mild or moderate COPD. In some embodiments, the change in baseline to peak FEV₁ for a subject having severe or very severe COPD is from about 40% to about 60% greater than for a subject having mild or moderate COPD. In some embodiments, the change in baseline to peak FEV₁ for a subject having severe or very severe COPD is about 55% greater than for a subject having mild or moderate COPD.

In some embodiments, administration of a pharmaceutical composition (e.g., comprising ensifentrine) to a subject having mild or moderate COPD provides a FEV₁ AUC₀₋₄ₕ (e.g., after 12 weeks of administration) of at least 0.13 L (130 mL). In some embodiments, administration of a pharmaceutical composition (e.g., comprising ensifentrine) to a subject having mild or moderate COPD provides a FEV₁ AUC₀₋₄ₕ (e.g., after 12 weeks of administration) of at least 0.14 L (e.g., 0.15 L, 0.16 L). In some embodiments, administration of a pharmaceutical composition (e.g., comprising ensifentrine) to a subject having mild or moderate COPD provides a FEV₁ AUC₀₋₄ₕ (e.g., after 12 weeks of administration) of at most 0.19 L (190 mL). In some embodiments, administration of a pharmaceutical composition (e.g., comprising ensifentrine) to a subject having mild or moderate COPD provides a FEV₁ AUC₀₋₄ₕ (e.g., after 12 weeks of administration) of at most 0.18 L (e.g., 0.17 L, 0.16 L). In some embodiments, administration of a pharmaceutical composition (e.g., comprising ensifentrine) to a subject having mild or moderate COPD provides a FEV₁ AUC₀₋₄ₕ (e.g., after 12 weeks of administration) of from about 0.13 L to about 0.19 L. In some embodiments, administration of a pharmaceutical composition (e.g., comprising ensifentrine) to a subject having mild or moderate COPD provides a FEV₁ AUC₀₋₄ₕ (e.g., after 12 weeks of administration) of about 0.14 L to about 0.18 L. In some embodiments, administration of a pharmaceutical composition (e.g., comprising ensifentrine) to a subject having mild or moderate COPD provides a FEV₁ AUC₀₋₄ₕ (e.g., after 12 weeks of administration) of about 0.16 L (160 mL).

In some embodiments, administration of a pharmaceutical composition (e.g., comprising ensifentrine) to a subject having severe or very severe COPD provides a FEV₁ AUC₀₋₄ₕ (e.g., after 12 weeks of administration) of at least 0.07 L (70 mL). In some embodiments, administration of a pharmaceutical composition (e.g., comprising ensifentrine) to a subject having severe or very severe COPD provides a FEV₁ AUC₀₋₄ₕ (e.g., after 12 weeks of administration) of at least 0.08 L (e.g., 0.09 L, 0.1 L). In some embodiments, administration of a pharmaceutical composition (e.g., comprising ensifentrine) to a subject having severe or very severe COPD provides a FEV₁ AUC₀₋₄ₕ (e.g., after 12 weeks of administration) of at most 0.14 L (140 mL). In some embodiments, administration of a pharmaceutical composition (e.g., comprising ensifentrine) to a subject having severe or very severe COPD provides a FEV₁ AUC₀₋₄ₕ (e.g., after 12 weeks of administration) of at most 0.13 L (e.g., 0.12 L, 0.11 L, 0.1 L). In some embodiments, administration of a pharmaceutical composition (e.g., comprising ensifentrine) to a subject having severe or very severe COPD provides a FEV₁ AUC₀₋₄ₕ (e.g., after 12 weeks of administration) of about 0.07 L to about 0.14 L. In some embodiments, administration of a pharmaceutical composition (e.g., comprising ensifentrine) to a subject having severe or very severe COPD provides a FEV₁ AUC₀₋₄ₕ (e.g., after 12 weeks of administration) of about 0.08 L to about 0.12 L. In some embodiments, administration of a pharmaceutical composition (e.g., comprising ensifentrine) to a subject having severe or very severe COPD provides a FEV₁ AUC₀₋₄ₕ (e.g., after 12 weeks of administration) of about 0.1 L (e.g., 100 mL).

In some embodiments, the FEV₁ AUC₀₋₄ₕ for a subject having mild or moderate COPD is at least 40% greater than for a subject having severe or very severe COPD. In some embodiments, the FEV₁ AUC₀₋₄ₕ for a subject having mild or moderate COPD is at most 70% greater than for a subject having severe or very severe COPD. In some embodiments, the FEV₁ AUC₀₋₄ₕ for a subject having mild or moderate COPD is from about 50% to about 70% greater than for a subject having severe or very severe COPD. In some embodiment, the FEV₁ AUC₀₋₄ₕ for a subject having mild or moderate COPD is about 60% greater than for a subject having severe or very severe COPD.

In some embodiments, the FEV₁ AUC₀₋₄ₕ for a subject having severe or very severe COPD is at least 40% greater than for a subject having mild or moderate COPD. In some embodiments, the FEV₁ AUC₀₋₄ₕ for a subject having severe or very severe COPD is at most 70% greater than for a subject having mild or moderate COPD. In some embodiments, the FEV₁ AUC₀₋₄ₕ for a subject having severe or very severe COPD is from about 50% to about 70% greater than for a subject having mild or moderate COPD. In some embodiment, the FEV₁ AUC₀₋₄ₕ for a subject having severe or very severe COPD is about 60% greater than for a subject having mild or moderate COPD.

FEV₁ as described herein may be measured using spirometry, which the skilled artisan would understand as a well standardized, easy to perform, and consistent reproducible measure of pulmonary function.

### Renal Impairment

In some embodiments, provided herein are methods of treating COPD in subjects having varying levels of renal impairment. In some embodiments, provided herein are methods of treating COPD in subjects having moderate renal impairment. Renal impairment of a subject may impact the clearance rate of ensifentrine, resulting in modified PK parameters in comparison to healthy subjects. In some embodiments, the methods comprise administering to the subject (e.g., a therapeutically effective amount of) ensifentrine. In some embodiments, the methods comprise administering to the subject a pharmaceutical composition provided herein.

Renal impairment of a subject as described herein is determined based on the absolute glomerular filtrate rate (aEGFR) of the subject. For example, individuals with normal renal function to mild renal impairment exhibit aEGFR of greater than or equal to 60 mL/min. Individuals with moderate renal impairment exhibit aEGFR of less than or equal to 60 mL/min. Individuals with normal renal function may exhibit aEGFR of greater than or equal to 90 mL/min.

In some embodiments, subjects with moderate renal impairment exhibit modified PK parameters, as compared to healthy subjects or those with mild renal impairment. In some embodiments, the modified PK parameters comprise one or more of a mean Cₘₐₓ, a Tₘₐₓ, or an AUC₀₋ₜₐᵤ.

In some embodiments, administering the pharmaceutical compositions (e.g., comprising ensifentrine) provided herein provide a blood plasma concentration of ensifentrine, different in subjects with moderate renal impairment as compared to healthy subjects or those with mild renal impairment (e.g., such as described in Example 1). In some embodiments, a pharmaceutical composition (e.g., comprising ensifentrine) provided herein provide a blood plasma concentration of ensifentrine comprising a mean Cₘₐₓ of from about 500 pg/mL to about 700 pg/mL in subjects having moderate renal impairment. In some embodiments, the mean Cₘₐₓ is from about 550 pg/mL to about 650 pg/mL in subjects having moderate renal impairment. In some embodiments, the mean Cₘₐₓ is about 604 pg/mL in subjects having moderate renal impairment. In some embodiments, the mean Cₘₐₓ is at least 400 pg/mL (e.g., at least 450 pg/mL, at least 500 pg/mL, at least 550 pg/mL, or at least 600 pg/mL) in subjects having moderate renal impairment. In some embodiments, the mean Cₘₐₓ is at least 550 pg/mL in individuals having moderate renal impairment. In some embodiments, the mean Cₘₐₓ is at most 750 pg/mL (e.g., at most 700 pg/mL, at most 650 pg/mL) in individuals having moderate renal impairment. In some embodiments, the mean Cₘₐₓ is at most 650 pg/mL in individuals having moderate renal impairment.

In some embodiments, the pharmaceutical compositions (e.g., comprising ensifentrine) provided herein provide a blood plasma concentration of ensifentrine comprising a mean Cₘₐₓ of from about 173 mL/min to about 1330 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of from about 150 mL/min to about 1400 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of from about 400 mL/min to about 700 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of at least 100 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of at least 200 mL/min (e.g., 300 mL/min, 400 mL/min, or 500 mL/min) in healthy subjects or those with mild renal impairment. In some embodiments, a pharmaceutical composition (e.g., comprising ensifentrine) provided herein provides a blood plasma concentration of ensifentrine comprising a mean Cₘₐₓ of at most 1000 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of at most 900 mL/min (e.g., 800 mL/min, 700 mL/min, 600 mL/min, or 500 mL/min) in healthy subjects or those with mild renal impairment. In some embodiments, a pharmaceutical composition (e.g., comprising ensifentrine) provided herein provides a blood plasma concentration of ensifentrine comprising a mean Cₘₐₓ of about 500 mL/min in healthy subjects or those with mild renal impairment. In some embodiments, a pharmaceutical composition (e.g., comprising ensifentrine) provided herein provides a blood plasma concentration of ensifentrine comprising a mean Cₘₐₓ of 522 mL/min in healthy subjects or those with mild renal impairment.

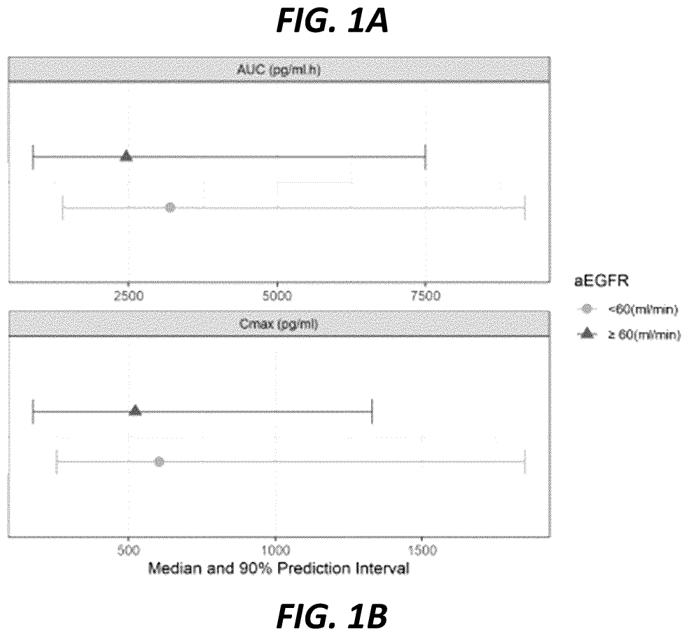

In certain embodiments, administering the pharmaceutical compositions to a subject having moderate renal impairment provide a blood plasma concentration comprising a mean Cₘₐₓ higher than that of individuals having normal renal function to mild renal impairment. In some embodiments, the mean Cₘₐₓ is at least 10% higher than a Cₘₐₓ for a subject administered the pharmaceutical composition not having moderate renal impairment. In some embodiments, the mean Cₘₐₓ is at least 5% higher (e.g., at least 6% higher, at least 7% higher, at least 8% higher, or at least 9% higher) in a subject having moderate renal impairment than a Cₘₐₓ for a subject administered the pharmaceutical composition not having moderate renal impairment. In some embodiments, the mean Cₘₐₓ is at most 15% higher (e.g., at most 14% higher, at most 13% higher, at most 12% higher, at most 11% higher) in a subject having moderate renal impairment than a Cₘₐₓ for a subject administered the pharmaceutical composition not having moderate renal impairment. In some embodiments, the mean Cₘₐₓ is about 16% higher in a subject having moderate renal impairment than a Cₘₐₓ for a subject administered the pharmaceutical composition not having moderate renal impairment. In some instances, the Cₘₐₓ for a subject having moderate renal impairment and a subject having normal renal function to mild renal impairment is shown in FIG. 1B.

In certain embodiments, administering the pharmaceutical compositions to a subject having normal renal function to mild renal impairment provide a blood plasma concentration comprising a mean Cₘₐₓ higher than that of individuals having moderate renal impairment. In some embodiments, the mean Cₘₐₓ is at least 10% higher than a Cₘₐₓ for a subject administered the pharmaceutical composition not having normal renal function to mild renal impairment. In some embodiments, the mean Cₘₐₓ is at least 5% higher (e.g., at least 6% higher, at least 7% higher, at least 8% higher, or at least 9% higher) in a subject having normal renal function to mild renal impairment than a Cₘₐₓ for a subject administered the pharmaceutical composition not having normal renal function to mild renal impairment. In some embodiments, the mean Cₘₐₓ is at most 15% higher (e.g., at most 14% higher, at most 13% higher, at most 12% higher, at most 11% higher) in a subject having normal renal function to mild renal impairment than a Cₘₐₓ for a subject administered the pharmaceutical composition not having normal renal function to mild renal impairment.

In some embodiments, administering the pharmaceutical compositions (e.g., comprising ensifentrine) provided herein provide a blood plasma concentration of ensifentrine comprising a mean AUC₀₋ₜₐᵤ (exposure) of from about 2600 pg/mL*h to about 3600 pg/mL*h in subjects having moderate renal impairment. In some embodiments, the pharmaceutical compositions provided herein provide a blood plasma concentration of ensifentrine comprising a mean AUC₀₋ₜₐᵤ (exposure) of from about 3000 pg/mL*h to about 3400 pg/mL*h. In some embodiments, the pharmaceutical compositions provided herein provide a blood plasma concentration of ensifentrine comprising a mean AUC₀₋ₜₐᵤ (exposure) of at least 2500 pg/mL*h (e.g., at least 2600 pg/mL*h, at least 2800 pg/mL*h, at least 3000 pg/mL*h, or at least 3100 pg/mL*h) in subjects having moderate renal impairment. In some embodiments, the pharmaceutical compositions provided herein provide a blood plasma concentration of ensifentrine comprising a mean AUC₀₋ₜₐᵤ (exposure) of about 3200 pg/mL*h in subjects having moderate renal impairment. In some embodiments, the pharmaceutical compositions provided herein provide a blood plasma concentration of ensifentrine comprising a mean AUC₀₋ₜₐᵤ (exposure) of at most 3750 pg/mL*h (e.g., at most 3700 pg/mL*h, at most 3600 pg/mL*h, at most 3500 pg/mL*h, at most 3400 pg/mL*h, or at most 3300 pg/mL*h) in subjects having moderate renal impairment.

In some embodiments, administering the pharmaceutical compositions provided herein provide a blood plasma concentration of ensifentrine comprising a mean AUC₀₋ₜₐᵤ (exposure) of about 882 pg/mL*h to about 7500 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ(exposure) of about 800 pg/mL*h to about 7500 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ (exposure) of at least 800 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ(exposure) of at least 900 pg/mL*h (e.g., 1200 pg/mL*h, 1400 pg/mL*h, 1600 pg/mL*h, 2000 pg/mL*h, 2200 pg/mL*h, or 2400 pg/mL*h) in healthy subjects or subjects with mild renal impairment. In some embodiments, administering the pharmaceutical compositions provided herein provide a blood plasma concentration of ensifentrine comprising a mean AUC₀₋ₜₐᵤ(exposure) of at most 8000 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ (exposure) of at most 8000 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ (exposure) of at most 7000 pg/mL*h (e.g., 6000 pg/mL*h, 5000 pg/mL*h, 4000 pg/mL*h, 3000 pg/mL*h, or 2500 pg/mL*h) in healthy subjects or subjects with mild renal impairment. In some embodiments, administering the pharmaceutical compositions provided herein provide a blood plasma concentration of ensifentrine comprising a mean AUC₀₋ₜₐᵤ (exposure) of about 2500 pg/mL*h in healthy subjects or subjects with mild renal impairment. In some embodiments, administering the pharmaceutical compositions provided herein provide a blood plasma concentration of ensifentrine comprising a mean AUC₀₋ₜₐᵤ (exposure) of 2450 pg/mL*h in healthy subjects or subjects with mild renal impairment.

In some embodiments, administering the pharmaceutical compositions provided herein provides a blood plasma concentration of ensifentrine comprising a mean AUC₀₋ₜₐᵤ (exposure) in subjects with moderate renal impairment different from the AUC₀₋ₜₐᵤ of individuals not having moderate renal impairment (e.g., having normal renal function or mild renal impairment). In some embodiments, the pharmaceutical compositions provided herein provide a blood plasma concentration of ensifentrine comprising a mean AUC₀₋ₜₐᵤ (exposure) that is at least 25% higher in subjects with moderate renal impairment than subjects without moderate renal impairment (e.g., normal renal function or mild renal impairment). In some embodiments, the pharmaceutical compositions provided herein provide a blood plasma concentration of ensifentrine comprising a mean AUC₀₋ₜₐᵤ (exposure) that is at least 10% higher (e.g., 12% higher, 14% higher, 16% higher, 18% higher, or 20% higher) in subjects with moderate renal impairment than subjects without moderate renal impairment (e.g., normal renal function or mild renal impairment). In some embodiments, the pharmaceutical compositions provided herein provide a blood plasma concentration of ensifentrine comprising a mean AUC₀₋ₜₐᵤ (exposure) that is at most 35% higher (e.g., 30% higher, 28% higher, 27% higher, or 26% higher) in subjects with moderate renal impairment than subjects without moderate renal impairment (e.g., normal renal function or mild renal impairment). In some instances, the AUC₀₋ₜₐᵤ for an individual having moderate renal impairment and an individual having normal renal function to mild renal impairment is shown in FIG. 1A.

In some embodiments, administering the pharmaceutical compositions provided herein provides a blood plasma concentration of ensifentrine comprising a mean AUC₀₋ₜₐᵤ (exposure) in subjects with normal renal function to mild renal impairment different from the AUC₀₋ₜₐᵤ of individuals not having normal renal function to mild renal impairment (e.g., having normal renal function or mild renal impairment). In some embodiments, the pharmaceutical compositions provided herein provide a blood plasma concentration of ensifentrine comprising a mean AUC₀₋ₜₐᵤ (exposure) that is at least 25% higher in subjects with normal renal function to mild renal impairment than subjects without normal renal function to mild renal impairment (e.g., normal renal function or mild renal impairment). In some embodiments, the pharmaceutical compositions provided herein provide a blood plasma concentration of ensifentrine comprising a mean AUC₀₋ₜₐᵤ (exposure) that is at least 10% higher (e.g., 12% higher, 14% higher, 16% higher, 18% higher, or 20% higher) in subjects with normal renal function to mild renal impairment than subjects without normal renal function to mild renal impairment (e.g., normal renal function or mild renal impairment). In some embodiments, the pharmaceutical compositions provided herein provide a blood plasma concentration of ensifentrine comprising a mean AUC₀₋ₜₐᵤ (exposure) that is at most 35% higher (e.g., 30% higher, 28% higher, 27% higher, or 26% higher) in subjects with normal renal function to mild renal impairment than subjects without normal renal function to mild renal impairment.

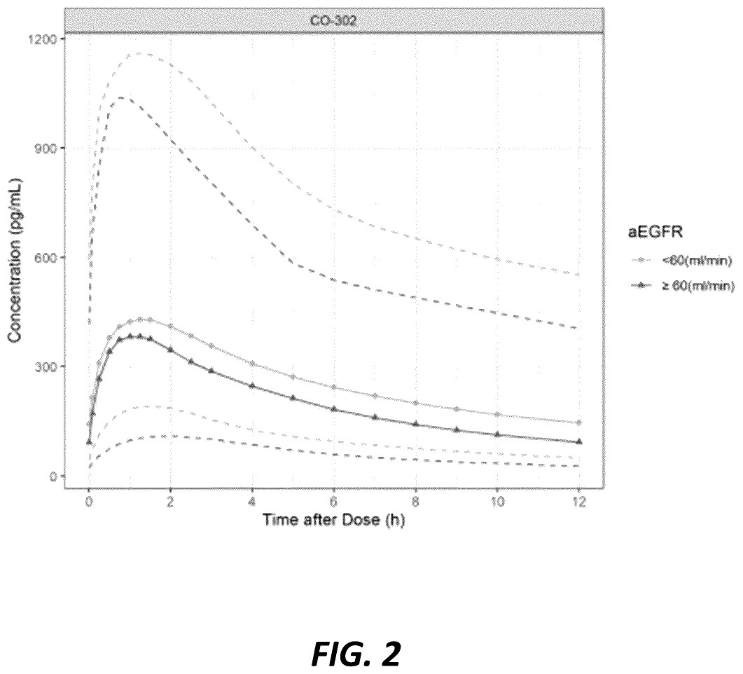

In some embodiments, administering the pharmaceutical compositions provided herein provide a blood plasma concentration of ensifentrine comprising a mean Tₘₐₓ in subjects with moderate renal impairment that differs from subjects without moderate renal impairment (e.g., with normal renal function or mild renal impairment). In some embodiments, the pharmaceutical compositions provided herein provide a blood plasma concentration of ensifentrine comprising a mean Tₘₐₓ in subjects with moderate renal impairment that is substantially the same as subjects without moderate renal impairment (e.g., with normal renal function or mild renal impairment). In some embodiments, the pharmaceutical compositions provided herein provide a blood plasma concentration of ensifentrine comprising a mean Tₘₐₓ of from about 1 h to about 2 h in subjects having moderate renal impairment. In some embodiments, the pharmaceutical compositions provided herein provide a blood plasma concentration of ensifentrine comprising a mean Tₘₐₓ of from about 1.2 h to about 1.7 h in subjects having moderate renal impairment. In some embodiments, the pharmaceutical compositions provided herein provide a blood plasma concentration of ensifentrine comprising a mean Tₘₐₓ of at least 0.8 hs (e.g., at least 0.9 h, at least 1 h, at least 1.2 h, at least 1.4 h) in subjects having moderate renal impairment. In some embodiments, the pharmaceutical compositions provided herein provide a blood plasma concentration of ensifentrine comprising a mean Tₘₐₓ of at most 2.2 h (e.g., at most 2 h, at most 1.8 h, at most 1.7 h, at most 1.6 h) in subjects having moderate renal impairment. In some embodiments, the mean Tₘₐₓ of an individual with moderate renal impairment and an individual without moderate renal impairment (e.g., normal renal function or mild renal impairment) is shown in FIG. 2.

The pharmacokinetic parameters of the blood plasma concentrations as provided herein may be measured at any suitable interval after administration of the pharmaceutical composition. In some embodiments, mean Cₘₐₓ, the mean AUC₀₋ₜₐᵤ, and the mean Tₘₐₓ are measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h). In some embodiments, mean Cₘₐₓ is measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h). In some embodiments, the mean AUC₀₋ₜₐᵤ is measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h). In some embodiments, the mean Tₘₐₓ is measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h).

### Hepatic Impairment

In some embodiments, provided herein are methods of treating COPD in subjects with varying liver function. In some embodiments, the liver function of a subject comprises healthy liver function, moderate hepatic impairment, or severe hepatic impairment. In some embodiments, provided herein are methods of treating COPD in subjects with a liver function comprising healthy liver function. In some embodiments, provided herein are methods of treating COPD in subjects with a liver function comprising moderate hepatic impairment. In some embodiments, provided herein are methods of treating COPD in subjects with a liver function comprising severe hepatic impairment.

In some instances, the liver function of a subject is determined using the Child Pugh score. The Child Pugh score classifies liver function (e.g., liver disease severity) according to the degree of ascites, the serum concentrations of bilirubin and albumin, the prothrombin time, and the degree of encephalopathy. A Child-Pugh score of 5-6 is considered Class A (well-compensated disease). A Child-Pugh score of 7-9 is considered Class B (significant functional compromise). A Child-Pugh score of 10-15 is considered Class C (decompensated disease). The skilled artisan would understand the Child-Pugh score to be an accepted method of defining liver function of a subject. Using the Child Pugh score, subjects are divided into groups including: healthy liver function, moderate hepatic impairment, or severe hepatic impairment. A subject determined to be Class A according to Child-Pugh scores may be an individual with normal liver function as used herein. A subject determined to be Class B according to Child-Pugh scores may be an individual with moderate hepatic impairment as used herein. A subject determined to be Class C according to Child-Pugh scores may be an individual with severe hepatic impairment as used herein.

In some embodiments, the pharmaceutical compositions provided herein provide a blood plasma concentration of ensifentrine comprising an AUC_{t,ss} (steady state area under the curve). In some instances, the AUC_{t,ss} is substantially the same regardless of the subject's liver function. In some instances, this is indicative that (1) patients that have moderate hepatic impairment or severe hepatic impairment may respond to ensifentrine as a treatment for COPD, and (2) that the ensifentrine compositions described herein are safe for subjects with varying levels of hepatic impairment ranging from healthy to severe hepatic impairment.

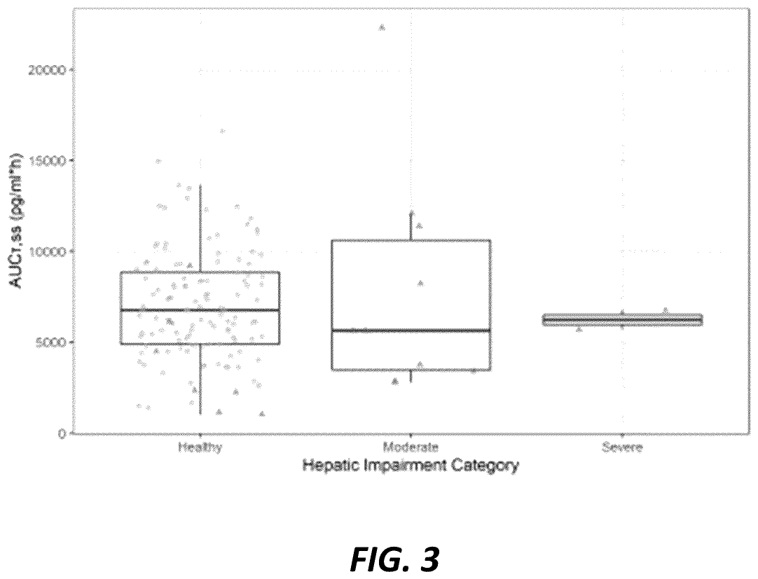

In some embodiments, administering the pharmaceutical compositions provides a blood plasma concentration comprising an AUC_{t,ss}, such as described in Example 1 and FIG. 3. In some embodiments, the AUC_{t,ss} is from about 5000 pg/mL*h to about 10000 pg/mL*h. In some embodiments, the AUC_{t,ss} is from about 5000 pg/mL*h to about 7500 pg/mL*h. In some embodiments, the AUC_{t,ss} is a least 4000 pg/mL*h (e.g., at least 4200 pg/mL*h, at least 4500 pg/mL*h, at least 4700 pg/mL*h, at least 4900 pg/mL*h, or at least 5000 pg/mL*h). In some embodiments, the AUC_{t,ss} is at most 10000 pg/mL*h (e.g., at most 9500 pg/mL*h, at most 9000 pg/mL*h, at most 8500 pg/mL*h, at most 8000 pg/mL*h, or at most 7500 pg/mL*h). In some embodiments, the AUC_{t,ss} of a healthy subject is not substantially different than the AUC_{t,ss} of a subject with moderate hepatic impairment. In some embodiments, the AUC_{t,ss} of a healthy subject is not substantially different than the AUC_{t,ss} of a subject with severe hepatic impairment. In some embodiments, the AUC_{t,ss} of a subject with moderate hepatic impairment is not substantially different than the AUC_{t,ss} of a subject with severe hepatic impairment.

In some embodiments, administering the pharmaceutical compositions provides a blood plasma concentration for healthy subjects comprising an AUC_{t,ss}, such as described in Example 1 and FIG. 3. In some embodiments, the AUC_{t,ss} for healthy subjects is from about 5000 pg/mL*h to about 10000 pg/mL*h. In some embodiments, the AUC_{t,ss} for healthy subjects is from about 5000 pg/mL*h to about 7500 pg/mL*h. In some embodiments, the AUC_{t,ss} for healthy subjects is a least 4000 pg/mL*h (e.g., at least 4200 pg/mL*h, at least 4500 pg/mL*h, at least 4700 pg/mL*h, at least 4900 pg/mL*h, or at least 5000 pg/mL*h). In some embodiments, the AUC_{t,ss} for healthy subjects is at most 10000 pg/mL*h (e.g., at most 9500 pg/mL*h, at most 9000 pg/mL*h, at most 8500 pg/mL*h, at most 8000 pg/mL*h, or at most 7500 pg/mL*h). In some embodiments, the AUC_{t,ss} for healthy subjects is about 7000 pg/mL*h.

In some embodiments, administering the pharmaceutical compositions provides a blood plasma concentration for subjects with moderate hepatic impairment comprising an AUC_{t,ss}, such as described in Example 1 and FIG. 3. In some embodiments, the AUC_{t,ss} for a subject with moderate hepatic impairment is from about 4500 pg/mL*h to about 11000 pg/mL*h. In some embodiments, the AUC_{t,ss} for a subject with moderate hepatic impairment is a least 4000 pg/mL*h (e.g., at least 4200 pg/mL*h, at least 4500 pg/mL*h, at least 4700 pg/mL*h, at least 4900 pg/mL*h, or at least 5000 pg/mL*h). In some embodiments, the AUC_{t,ss} for a subject with moderate hepatic impairment is at most 12000 pg/mL*h (e.g., at most 11000 pg/mL*h, at most 9500 pg/mL*h, at most 9000 pg/mL*h, at most 8500 pg/mL*h, at most 8000 pg/mL*h, or at most 7500 pg/mL*h). In some embodiments, the AUC_{t,ss} for a subject with moderate hepatic impairment is about 6000 pg/mL*h. In some embodiments, the AUC_{t,ss} for a subject with moderate hepatic impairment is about 5500 pg/mL*h.

In some embodiments, administering the pharmaceutical compositions provides a blood plasma concentration for subjects with severe hepatic impairment comprising an AUC_{t,ss}, such as described in Example 1 and FIG. 3. In some embodiments, the AUC_{t,ss} for a subject with severe hepatic impairment is from about 5000 pg/mL*h to about 7500 pg/mL*h. In some embodiments, the AUC_{t,ss} for a subject with severe hepatic impairment is from about 5500 pg/mL*h to about 7000 pg/mL*h. In some embodiments, the AUC_{t,ss} for a subject with severe hepatic impairment is a least 5000 pg/mL*h (e.g., at least 5200 pg/mL*h, at least 5500 pg/mL*h, at least 5700 pg/mL*h, at least 5900 pg/mL*h, or at least 6000 pg/mL*h). In some embodiments, the AUC_{t,ss} for a subject with severe hepatic impairment is at most 7500 pg/mL*h (e.g., at most 7000 pg/mL*h, at most 6500 pg/mL*h, or at most 6000 pg/mL*h. In some embodiments, the AUC_{t,ss} for a subject with severe hepatic impairment is about 6500 pg/mL*h. In some embodiments, the AUC_{t,ss} for a subject with severe hepatic impairment is about 7000 pg/mL*h.

### Trough Lung Function

In some embodiments, provided herein are methods of increasing trough lung function in a subject suffering from COPD, the method comprising administering to the subject (e.g., a therapeutically effective amount of) ensifentrine. In some embodiments, provided herein are methods of increasing trough lung function in a subject suffering from COPD, the method comprising administering to the subject a pharmaceutical composition (e.g., comprising ensifentrine) described elsewhere herein.

In some instances, improving trough lung function comprises improvements in morning and night trough lung function. In some embodiments, improving trough lung function comprises improvements in morning trough lung function. In some embodiments, improving night trough lung function.

In some embodiments, provided herein are methods of increasing trough lung function in a subject suffering from COPD, wherein the subject has healthy liver function, moderate hepatic impairment, or severe hepatic impairment. In some embodiments, provided herein are methods of increasing trough lung function in a subject suffering from COPD, wherein the subject has healthy liver function. In some embodiments, provided herein are methods of increasing trough lung function in a subject suffering from COPD, wherein the subject has moderate hepatic impairment. In some embodiments, provided herein are methods of increasing trough lung function in a subject suffering from COPD, wherein the subject has severe hepatic impairment. In some embodiments, the hepatic function of the subject is determined by the Child-Pugh score as described hereinabove.

In some embodiments, provided herein are methods of increasing trough lung function in a subject suffering from COPD, wherein the subject has healthy renal function or mild renal impairment. In some embodiments, provided herein are methods of increasing trough lung function in a subject suffering from COPD, wherein the subject has moderate renal impairment.

In some embodiments, provided herein is a method of treating COPD in a subject comprising administering (e.g., a therapeutically effective amount of) ensifentrine, wherein the subject is susceptible to disturbed sleep. In some embodiments, provided herein is a method of treating COPD in a subject comprising administering a pharmaceutical composition described elsewhere herein, wherein the subject is susceptible to disturbed sleep.

In some instances, an increase in trough lung function in a subject is determined by measuring an increase in trough FEV₁ (e.g., the FEV₁ of the subject shortly before administration of the compound as part of the maintenance therapy). The improvement in trough lung function may for example arise from a change in maintenance therapy drug.

In some embodiments, the methods herein comprise increasing morning trough lung function (e.g., trough lung function following sleep). Morning trough lung function may be measured by determining the FEV₁ of the subject shortly before the morning administration of the compound as part of the maintenance therapy. In some embodiments, FEV₁ may be measured less than an hour before the morning administration of the ensifentrine. Morning trough FEV₁ may be the FEV₁ as measured between 11.5 and 12 h following the prior evening dose.

In some embodiments, improvement in trough lung function can improve sleep in a COPD subject. In some instances, this may be important in subjects susceptible to disturbed sleep. Subjects susceptible to disturbed sleep typically suffer from a condition that directly affects their ability to sleep (e.g., insomnia or sleep apnea) or from a condition that indirectly makes it harder to sleep (e.g., skin condition such as psoriasis that irritate the skin and make it harder for a patient to fall asleep or remain asleep).

As used herein, a “patient susceptible to disturbed sleep” is typically a patient suffering from one or more disease or condition selected from obesity, insomnia, sleep apnea, narcolepsy, restless leg syndrome, REM sleep behavior disorder, circadian rhythm sleep disorders parasomnias, depression, anxiety, psoriasis, dermatitis, eczema, or urticaria. In some embodiments, the ensifentrine or pharmaceutical composition comprising ensifentrine may be used in treating COPD in a subject suffering from COPD and sleep apnea. In some embodiments, the ensifentrine or pharmaceutical composition comprising ensifentrine may be used in treating COPD in a subject suffering from COPD and a skin condition such as psoriasis, dermatitis, eczema, or urticaria.

In some embodiments, the improvement in trough lung function can (e.g., significantly) assist a COPD subject in exercising. In some instances, the patient is suffering from obesity.

### COPD Exacerbations

In some embodiments, provided herein are methods of treating the frequency and/or severity of COPD exacerbations in a subject suffering from COPD, the method comprising administering to the subject (e.g., a therapeutically effective amount of) ensifentrine comprised in a pharmaceutical composition provided herein. In some embodiments, provided herein is a method of treating the frequency of COPD exacerbations in a subject, the method comprising administering to the subject (e.g., a therapeutically effective amount of) ensifentrine comprised in a pharmaceutical composition provided herein. In some embodiments, provided herein is a method of treating the severity of COPD exacerbations in a subject, the method comprising administering to the subject (e.g., a therapeutically effective amount of) ensifentrine comprised in a pharmaceutical composition provided herein. Also provided herein in some embodiments is a method of increasing the time to first COPD exacerbation in a subject suffering from COPD, comprising administering (e.g., a therapeutically effective amount of) ensifentrine comprised in a pharmaceutical composition provided herein.

COPD exacerbations can be life-threatening and have been linked to comorbid conditions. They may also be associated with irreversible increases in airflow obstruction, which may lead to a higher frequency of hospitalization and increased risk of death. Exacerbations are also important events in a patient's COPD progression since they have a negative impact on health-related quality of life and may be associated with an accelerated decline in lung function and increased mortality. As such, methods of treating/decreasing the frequency of COPD exacerbations are desirable.

In some embodiments, provided herein is a method of treating COPD in a subject, comprising administering to the subject (e.g., a therapeutically effective amount of) ensifentrine or a pharmaceutically acceptable salt thereof, wherein the subject is susceptible to COPD exacerbations. In some embodiments, provided herein is a method of treating COPD in a subject, comprising administering to the subject a pharmaceutical composition provided elsewhere herein, wherein the subject is susceptible to COPD exacerbations.

In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has normal liver function, moderate hepatic impairment, or severe hepatic impairment. In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has normal liver function. In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has moderate hepatic impairment. In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has severe hepatic impairment. In some embodiments, the hepatic function of the subject is determined by the Child-Pugh score as described elsewhere herein.

In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has normal renal function to mild renal impairment or moderate renal impairment. In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has normal renal function to mild renal impairment. In some embodiments, provided herein is a method of treating COPD in a subject, comprising administering to the subject a pharmaceutical composition (e.g., comprising ensifentrine) as provided elsewhere herein, wherein the subject is susceptible to COPD exacerbations and has moderate renal impairment.

In some embodiments, the ensifentrine or pharmaceutically acceptable salt thereof may decrease the frequency and/or severity of COPD exacerbations in a subject suffering from COPD. In some embodiments, the ensifentrine or pharmaceutically acceptable salt thereof may decrease the frequency of COPD exacerbations. For example, the subject may suffer two or fewer (e.g., one or zero) COPD exacerbations per year while being treated with the pharmaceutical compositions provided herein (e.g., comprising ensifentrine), for example as a maintenance therapy. The number of COPD exacerbations experienced by the subject per year during treatment with the pharmaceutical compositions provided herein (e.g., comprising ensifentrine) may be one to three fewer than the number of COPD exacerbations experienced by the subject per year prior to treatment with ensifentrine.

In some embodiments, administration with a pharmaceutical composition provided herein reduces the rate of COPD exacerbations by from about 30% to about 50%. In some embodiments, administration with a pharmaceutical composition provided herein reduces the rate of COPD exacerbations by from about 35% to about 45%. In some embodiments, administration with a pharmaceutical composition provided herein reduces the rate of COPD exacerbations by at least 25%. In some embodiments, administration with a pharmaceutical composition provided herein reduces the rate of COPD exacerbations by at least 30% (e.g., 32%, 34%, 36%, 38%, 40%, or 42%). In some embodiments, administration with a pharmaceutical composition provided herein reduces the rate of COPD exacerbations by at most 50% (e.g., 48%, 46%, 44%, 42%, or 40%). In some embodiments, administration with a pharmaceutical composition provided herein reduces the rate of COPD exacerbations by about 36%, 43%, or 44%. In some embodiments, administration with a pharmaceutical composition provided herein reduces the rate of COPD exacerbations by about 40%. In some embodiments, administration with a pharmaceutical composition provided herein reduces the rate of COPD exacerbations by about 36%. Reduction of COPD exacerbations may occur for any subgroup of subjects, including subjects with varying renal or hepatic impairment such as described elsewhere herein.

In some embodiments, administration with a pharmaceutical composition provided herein may provide longer times to a first moderate or severe COPD exacerbation (e.g., such as compared to subjects administered with a placebo). In some embodiments, administration with a pharmaceutical composition provided herein may provide at least a 25% reduction in risk of a COPD exacerbation (e.g., such as over 24 weeks of treatment). In some embodiments, administration with a pharmaceutical composition provided herein may provide at least a 30% (e.g., 32%, 34%, 36%, or 38%) reduction in risk of a COPD exacerbation. In some embodiments, administration with a pharmaceutical composition provided herein may provide at most a 60% reduction in COPD risk (e.g., 58%, 56%, 52%, 50%, 46%, or 42%). In some embodiments, administration with a pharmaceutical composition provided herein may provide a reduction in COPD exacerbation risk of about 38%. In some embodiments, administration with a pharmaceutical composition provided herein may provide a reduction in COPD exacerbation risk of about 52% (e.g., such as over 48 weeks of administration). In some instances, the reduction of risk of a COPD exacerbation may be determined by Cox's proportional hazard test and/or by log-rank test.

In some embodiments, the ensifentrine or pharmaceutically acceptable salt thereof may increase the time to a first COPD exacerbation in a subject. In some instances, the subject may not have yet experienced a COPD exacerbation and the ensifentrine may increase the time until the subject experiences a first COPD exacerbation (e.g., the first COPD exacerbation is delayed). The ensifentrine or pharmaceutically acceptable salt thereof may accordingly reduce the risk of COPD exacerbations in a subject with COPD.

The COPD exacerbations as described herein may comprise one or more of dyspnea (breathlessness), increased coughing, increased sputum volume, sputum purulence, wheezing, sore throat, a cold, and fever. Sputum purulence is a change in the color of spontaneously expectorated samples from uncolored to yellow-green. The COPD exacerbation may last for at least one day or at least two days. In some embodiments, the COPD exacerbation lasts for one day. In some embodiments, the COPD exacerbation lasts for at least one day. In some embodiments, the COPD exacerbation lasts for at least two days.

In some embodiments, the methods of treating the frequency and/or severity of COPD exacerbations also treat dyspnea in the subject. In some embodiments, the methods of treating the frequency and/or severity of COPD exacerbations also decrease the frequency of coughing in the subject. In some embodiments, the methods of treating the frequency and/or severity of COPD exacerbations also decrease sputum volume in the subject. In some embodiments, the methods of treating the frequency and/or severity of COPD exacerbations also improve sputum purulence in the subject. In some embodiments, the methods of treating the frequency and/or severity of COPD exacerbations also improve wheezing in the subject. In some embodiments, the methods of treating the frequency and/or severity of COPD exacerbations also improve sore throat in the subject. In some embodiments, the methods of treating the frequency and/or severity of COPD exacerbations also improve symptoms of a cold in the subject. In some embodiments, the methods of treating the frequency and/or severity of COPD exacerbations also improve symptoms of a fever in the subject.

In some embodiments, COPD exacerbation comprises worsening of two or more of the following major symptoms for at least two consecutive days: dyspnea, sputum volume and sputum purulence. In some embodiments, COPD exacerbation comprises worsening of any one major symptom together with any one of the following minor symptoms for at least two consecutive days: sore throats, colds (nasal discharge and/or nasal congestion), fever (oral temperature >37.5° C.) without other cause and increased cough. In some instances, a COPD exacerbation comprises worsening of two or more of the major symptoms (dyspnea, sputum volume, and sputum purulence) for at least two consecutive days.

A COPD exacerbation may be a moderate COPD exacerbation or a severe COPD exacerbation. A moderate exacerbation is defined as worsening symptoms of COPD (as defined hereinabove) requiring a minimum of three days of treatment with oral/systemic corticosteroids and/or antibiotics. A severe exacerbation is defined as worsening symptoms of COPD (as defined hereinabove) requiring in-patient hospitalization. In some embodiments, the ensifentrine or pharmaceutically acceptable salt thereof reduces the severity of COPD exacerbations in a patient, and may be used in preventing severe COPD exacerbations in a subject. In some embodiments, the ensifentrine or pharmaceutically acceptable salt thereof reduces the severity of COPD exacerbations. In some embodiments, the ensifentrine or pharmaceutically acceptable salt there reduces the severity of COPD exacerbations. In some instances, the subject may experience no severe COPD exacerbations in the year following first administration of the compound.

In some embodiments, the pharmaceutical composition provided herein lengthens the time to a COPD exacerbation. In some embodiments, the pharmaceutical composition provided herein may increase the time to a COPD exacerbation by at least one week. In some embodiments, the pharmaceutical composition provided herein may increase the time to a COPD exacerbation by at least 2 weeks. In some embodiments, the pharmaceutical composition provided herein may increase the time to a COPD exacerbation by at least 1 month. In some embodiments, the pharmaceutical composition provided herein may increase the time to a COPD exacerbation by at least two months. In some embodiments, the pharmaceutical composition provided herein may increase the time to a COPD exacerbation by at least three, four, five, or six months.

A “subject susceptible to COPD exacerbations” as described herein may be a subject suffering from one or more co-morbidities (other than COPD). In some instances, said subject is suffering from one or more disease or condition selected from asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease or tuberculosis, optionally wherein the asthma is allergic asthma, steroid resistant asthma, severe asthma, or pediatric asthma. The patient susceptible to COPD exacerbation may have a chronic bronchitis etiology, may have impaired lung function (such as 30 to 70% predicted FEV₁) or may have COPD symptoms despite use of long-acting muscarinic receptor antagonist (LAMA) or a long-acting beta-adrenergic receptor agonist (LABA) therapy.

In some embodiments, the pharmaceutical compositions provided herein may be used to treat COPD in a subject suffering from both COPD and asthma. In some embodiments, the ensifentrine or pharmaceutically acceptable salt thereof may be used in treating a subject suffering from both COPD and pulmonary hypertension. In some embodiments, the pharmaceutical compositions provided herein may be used in treating a subject suffering from both COPD and bronchiectasis.

In some embodiments, risk factors for COPD exacerbation include: high serum immunoglobulin (Ig), previous tuberculosis, severe airflow obstruction, chest infection and one or more hospital admission for COPD exacerbations in the previous year.

In some embodiments, the subjects provided herein may have suffered one or more COPD exacerbations in the year preceding the first administration of ensifentrine or a pharmaceutically acceptable salt thereof. In some embodiments, the subject may have suffered two or more COPD exacerbations in the year preceding the first administration of a pharmaceutical composition provided herein. In some embodiments, the subject may have suffered from at least one severe COPD exacerbation (e.g., requiring hospital treatment) in the preceding year. The subject may have suffered from one or more COPD exacerbations in the six months preceding the first administration of the pharmaceutical composition provided herein, or one or more COPD exacerbations in the one month preceding the first administration of the pharmaceutical composition provided herein.

### Administration Methods

In some embodiments, the methods provided herein comprise administering to the subject a therapeutically effective amount of ensifentrine. In some embodiments, a therapeutically effective amount of ensifentrine comprises a dose of ensifentrine as described elsewhere herein. In some embodiments, the therapeutically effective amount of ensifentrine is comprised in a pharmaceutical composition provided herein.

In some embodiments, the methods provided herein comprise administering the pharmaceutical composition to the subject by inhalation. In some embodiments, a pharmaceutical composition comprising ensifentrine and one or more acceptable excipients or diluents is administered to a subject by inhalation. In some embodiments, administration by inhalation is completed using a nebulizer, pressurized metered dose inhaler (pMDI), or dry powder inhaler (DPI). In some embodiments, administration by inhalation is completed using a nebulizer. In some embodiments, administration by inhalation is completed using a pressurized metered dose inhaler (pMDI). In some embodiments, administration by inhalation is completed using a dry powder inhaler (DPI).

In some embodiments, the methods provided herein comprise administering the pharmaceutical composition by inhalation from a nebulizer. Nebulizers aerosolize a liquid pharmaceutical composition into an aerosol that is inhaled into a patient's respiratory tract. Non-limiting examples of nebulizers include a soft mist nebuliser, a vibrating mesh nebuliser, a jet nebuliser and an ultrasonic wave nebulizer. Suitable nebuliser devices include the Philips I-neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The ensifentrine may be inhaled via the nebulizer for from 1 to 15 minutes. The pharmaceutical composition may be inhaled via the nebulizer for from about 1 to about 15 minutes. In some embodiments, the pharmaceutical composition may be inhaled for at least 1 minute. In some embodiments, the pharmaceutical composition may be inhaled for about 1 minute to about 12 minutes, about 1 minute to about 8 minutes, about 1 minute to about 4 minutes, about 3 minutes to about 15 minutes, about 4 minutes to about 10 minutes, or about 6 minutes to about 12 minutes. In some embodiments, the pharmaceutical composition is inhaled via the nebulizer for about 5 to about 7 minutes. In some embodiments, the pharmaceutical composition is inhaled via the nebulizer for about 5 minutes. In some embodiments, the pharmaceutical composition is inhaled via the nebulizer for about 6 minutes. In some embodiments, the pharmaceutical composition is inhaled via the nebulizer for about 7 minutes.

### Dosing Regimens

The methods provided herein comprise administering the pharmaceutical composition to the subject at any frequency necessary to achieve therapeutic efficacy, as determined by the skilled artisan. In some embodiments, the pharmaceutical composition comprising ensifentrine is provided to the subject as a maintenance therapy. In some embodiments, the methods provided herein comprise administering the pharmaceutical composition to the subject once, twice, or three times per day, for instance twice or three times per day. In some embodiments, the methods comprise administering the pharmaceutical composition to the subject once per day. In some embodiments, the methods provided herein comprise administering the pharmaceutical composition to the subject twice per day (e.g., as a twice daily maintenance therapy). In some embodiments, the methods comprise administering the pharmaceutical composition to the subject three times per day. In some embodiments, the methods comprise administering the ensifentrine to the subject at least once per day. In some embodiments, the methods comprise administering a first dose of the pharmaceutical composition in the morning (e.g., within 3 h following waking) and a second dose of the pharmaceutical composition in the evening (e.g., within 3 h before bed). In some embodiments, morning and evening doses are administered from about 10 to about 14 h apart. In some embodiments, morning and evening doses are administered about 12 h apart.

The ensifentrine provided herein may be administered in any suitable therapeutically effective amount. In some embodiments, a daily dose of the ensifentrine is about 0.1 mg to about 20 mg. In some embodiments, the methods herein comprise administering a total daily dose of the ensifentrine of about 0.5 mg to about 10 mg. In certain embodiments, the total daily dose of the ensifentrine (e.g., ensifentrine free base) is about 5 mg to about 7 mg. In some embodiments, the total daily dose of the ensifentrine is about 6 mg per day. In some instances, the total daily dose of the ensifentrine may be 6.0 mg.

The methods provided herein may comprise administering the ensifentrine (e.g., or a pharmaceutical composition comprising ensifentrine) twice a day in two separate doses, which are the same or similar. In some instances, the method may comprise administering the compound to the subject twice a day in a first dose of from about 1 mg to about 5 mg and a second dose of from about 1 mg to about 5 mg. In certain embodiments, the method comprises administering the ensifentrine (e.g., or a pharmaceutical composition comprising ensifentrine) to the subject twice per day in a first dose of from about 2 mg to about 4 mg and a second dose of from about 2 mg to about 4 mg. In certain embodiments, the method comprises administering the ensifentrine (e.g., or a pharmaceutical composition comprising ensifentrine) to the subject twice per day in a first dose of about 3 mg and a second dose of about 3 mg.

In certain embodiments, the methods provided herein comprise administering two doses of about 3 mg of ensifentrine (e.g., or a pharmaceutical composition comprising ensifentrine) to the subject per day by inhalation. The method may comprise administering a dose of about 3 mg of the ensifentrine (e.g., or a pharmaceutical composition comprising ensifentrine) to the subject twice a day (e.g., 3 mg B.I.D.) by inhalation. In some embodiments, the method comprises administering by nebulizer a dose of about 3 mg of the ensifentrine (e.g., or a pharmaceutical composition comprising ensifentrine) to the subject twice per day. In some instances, each dose may comprise 3.0 mg of ensifentrine free base administered by nebulizer.

In some embodiments, the ensifentrine (e.g., or a pharmaceutical composition comprising ensifentrine) as provided herein is used as a maintenance therapy. In some embodiments, the methods provided herein comprise administering the pharmaceutical compositions to the subject at least once per day for at least 8 weeks. In some embodiments, the pharmaceutical compositions may be administered to the subject at least once per day for at least 16 weeks. In some embodiments, the pharmaceutical compositions may be administered to the subject at least once per day for at least 24 weeks. In some embodiments, the pharmaceutical compositions may be administered daily to the subject for at least 1 year. The methods provided herein may comprise administering the pharmaceutical compositions to the subject at least once every 24 h. In some instances, the method comprises administering the pharmaceutical compositions at least twice every 24 h, such as for at least 8 weeks, 16 weeks, or 24 weeks. In some embodiments, the pharmaceutical compositions are administered to the subject for any suitable period of time as determined by a skilled artisan.

In some embodiments, the ensifentrine is administered as a suspension formulation. In some embodiments, the ensifentrine is delivered as a dry powder, for instance a dry powder comprising particles comprising the ensifentrine and particles of a carrier such as lactose. In certain embodiments, the methods herein comprise administering an inhalable pharmaceutical composition comprising a suspension of particles of the ensifentrine in a diluent.

As described herein, the ensifentrine particles may be dispersed in a diluent (e.g., forming a suspension of particles in the diluent). A diluent may comprise water or a buffer (e.g., phosphate buffer). In some embodiments, a diluent comprises water. In some embodiments, a diluent comprises a buffer. In some embodiments, a diluent comprises a phosphate buffer.

In some embodiments, the ensifentrine particles may be dispersed in a sterile liquid vehicle. In some embodiments, the sterile liquid vehicle comprises one or more diluents. In some embodiments, the sterile liquid vehicle comprises a single diluent, such as water.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1: Ensifentrine Treatment of Subjects with Renal or Hepatic Impairment

A clinical study was conducted to determine the efficacy of ensifentrine in treating COPD compared with placebo. Ensifentrine was administered by nebulizer at a dose of 3 mg twice daily (BID) for 24 weeks. The study was a multicenter, randomized, double-blind, parallel group, placebo-controlled trial with around 800 patients and 5:3 randomization.

The study population included patients aged 40-80 years with moderate to severe COPD (FEV₁ 30%-70% p.n., FEV₁/forced vital capacity (FVC) ratio<0.7, with mMRC≥2). The randomization stratified (a) the use of stable background maintenance LAMA or LABA therapy use (approx. 50% yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC₀₋₁₂ₕ) over 4 h post-dose at week 12; morning trough FEV₁ at week 12; and other endpoints including moderate/severe COPD exacerbations frequency over 24 weeks.

### Methods

Baseline FEV₁ is the mean of the two measurements taken before study medication on the day of first dosing, i.e., ≤40 minutes and just prior to dosing, both pre-dose on day 1. Average FEV₁ AUC₀₋₁₂ₕ is defined as area under the curve over 12 h of the FEV₁, divided by 12 h.

Plasma concentrations of ensifentrine for the assessment of pharmacokinetic (PK) parameters were measured using validated bioanalytical methods for ensifentrine in human plasma with LLOQ of 5 pg/mL. Samples were taken on the following schedule:
Week 6: 1.0 h (±0.5 h) and 2.5 h (±0.5 h) (even sites) or 1.5 h and 4 h (±1 h) (odd sites);
Week 12: pre-dose (−0.5 h), 4 to 6 h, and 8 to 12 h (even sites) or 0.5 h (±0.25 h), 3 h (±0.5 h), and 6 to 8 h (odd sites);
Week 24: pre-dose (−0.5 h) and 1.5 h (even sites) or pre-dose (−0.5 h) and 1 h (±0.5 h) (odd sites)

### Formulation

The investigational product and placebo were provided in 2.5 mL unit dose format in an ampule and administered via a nebulizer. The formulation of the investigational product (ensifentrine suspension formulation comprising crystalline polymorph Form I of ensifentrine) and placebo are shown in Table 2.

TABLE 2

| Constituent | Concentration (mg/mL) |
|---|---|
| Ensifentrine | 1.2 (for the active) |
| particles (RPL554) | or 0 (for Placebo) |
| Dv10 = 0.5 μm | |
| Dv50 = 1.5 μm | |
| Dv90 = 3.6 μm | |
| Polysorbate 20 | 0.50 |
| (Tween 20) | |
| Sorbitan Monolaurate | 0.05 |
| (Span 20) | |
| Sodium Dihydrogen | 0.744 |
| Phosphate Dihydrate | |
| Disodium Hydrogen | 0.853 |
| Phosphate Dihydrate | |
| Sodium Chloride | 8.60 |
| Water | q.s. to 1 mL |

### Results—Renal Impairment

The primary endpoint of average FEV₁ AUC₀₋₁₂ₕ at week 12 was met. The results are shown in Table 3.

TABLE 3

| Average FEV₁ (AUC)0-12 h | Ensifentrine | |
|---|---|---|
| post-dose at Week 12: | 3 mg | Placebo |
| Change from Baseline (CFB) | (n = 498) | (n = 291) |
| Mean Baseline FEV₁, L | 1.3323 | 1.2316 |
| CFB Least Squares Mean, | 48.2 (30.0, 66.5) | −45.7 (−69.7, |
| mL (95% CI) | | −21.6) |
| Placebo-corrected CFB LS | 93.9 (64.5, 123.3) | — |
| Mean Diff., mL (95% CI) | | |
| p-value | <0.0001 | — |

Based on the samples taken during the trial, PK data was modelled for COPD patients with varying levels of renal function:

### Model

A two-compartment model with first-order input and first-order elimination from the central compartment was fit to the plasma concentration-time data of ensifentrine using NONMEM® version 7.4.4. Different absorption functions and compartmental structures were explored. Inter-individual variability was added to apparent plasma clearance (CL/F), volume of the central and peripheral compartments (V2/F and V3/F respectively), intercompartmental clearance (OIF), rate of first-order absorption (Ka), relative bioavailability (F1). Residual variability was added using a proportional error model, with addition of inter-individual variability (IIV) on residual error and inter-occasion variability (IOV) added on F1.

The first-order conditional estimation method (FOCE) with interaction was used initially, which frequently encountered numerical issues with the minimization. For the covariate model building, considering the dataset consisted in a large part of sparse data and convergence issues with FOCE, the estimation method was switched to the stochastic approximation expectation maximization (SAEM) to obtain parameter estimates followed by importance sampling (IMP) to obtain the objective function value (OFV). A forward addition process and backward elimination process (using goodness of fit plots, magnitude of the effect, precision of estimates, and change in the OFV equivalent to p<0.05 and p<0.001, respectively) were conducted to evaluate the effect of covariates on CL/F, V2/F, Q/F, V3/F, Ka, and F1. The primary covariates of interest included: disease state, body weight, age, race, ethnicity, sex, renal function, liver function, liver function enzymes, dose and formulation. Additional exploratory covariates were to be evaluated only if the Empiric Bayes Estimate (EBE) versus covariate plots suggested that there was a moderate to large effect.

The final model was evaluated using goodness of fit plots, bootstrap analysis, and visual predictive checks (VPCs). Simulations were then conducted on selected covariates to further evaluate the clinical relevance of findings.

Results from Model

Subjects with normal renal function to mild renal impairment are defined as those individuals with an absolute estimated glomerular filtration rate (aEGFR) of greater than or equal to 60 mL/min. Subjects with moderate renal impairment are defined as those individuals with an aEGFR of less than 60 mL/min. The final popPK model was used to predict parameter estimates in a virtual population of 50000 subjects (per covariate condition) for simulation of ensifentrine PK following nebulization of 3 mg in a population reflecting the population in the trial with normal renal function or mild renal impairment, or with moderate renal impairment. Uncertainty in fixed effects and covariance matrix parameters as well as residual variability were included in the simulations. The parameter uncertainty was accounted for by random resampling (without replacement) of 500 sets of parameter estimates from the bootstrap (750 replicates) in R to create 500 NONMEM simulation control streams. From each set of parameter estimates, the PK profiles for 100 subjects (for each covariate condition) were simulated with IV and IOV from the covariance matrix and residual error in NONMEM. Individual subject concentrations were simulated using NONMEM version 7.4.3, and PK parameters were calculated using R version 3.4.0. The results are shown in Table 4 and in FIG. 1A-B and FIG. 2.

TABLE 4

| | | | Ratio |
|---|---|---|---|
| | Median (90%) prediction interval | | aEGFR < 60 |
| Parameter | aEGFR ≥ 60 | aEGFR < 60 | mL/min: |
| (units) | mL/min | mL/min | ≥60 mL/min |
| Cₘₐₓ (pg/mL) | 522 | 604 | 1.16 |
| | (173-1330) | (255-1850) | (0.489-3.55) |
| AUC (pg/mL*h) | 2450 | 3200 | 1.30 |
| | (882-7500) | (1390-9170) | (0.567-3.74) |

It has been found that ensifentrine can provide a statistically significant improvement in lung function in individuals with normal renal function to mild renal impairment, as well as subjects with moderate renal impairment. For subjects with moderate renal impairment, a Cₘₐₓ of 604 pg/mL and a mean AUC of 3200 pg/mL*h were achieved in patients with COPD. These values were lower than the mean Cₘₐₓ (852 pg/mL) and mean AUC (mean AUC_{0-∞} of 5929 pg/mL*h) observed following administration of the composition to healthy patients, as well as lower than patients with normal renal function to mild renal impairment, indicating lower systemic exposure of ensifentrine in COPD patients than in healthy patients or those with normal renal function to mild renal impairment.

### Results—Hepatic Impairment

The dose normalized steady-state AUCₜₐᵤ for a 3 mg dose in non-COPD subjects with and without hepatic impairment is shown in FIG. 3. As described in FIG. 3 the AUC_{tau,ss} of healthy subjects was from about 5000 pg/mL*h to about 9000 pg/mL*h. The AUC_{tau,ss} of subjects with moderate hepatic impairment was about 4000 pg/mL*h to about 11000 pg/mL*h. The AUC_{tau,ss} of subjects with severe hepatic impairment was about 5500 pg/mL*h to about 6500 pg/mL*h. Exposure (AUC) in subjects with moderate (n=10) or severe (n=2) hepatic impairment was comparable to healthy subjects. The highest dose normalized AUC_{tau,ss} observed in this subset was for a subject with moderate hepatic impairment, but this was not the highest exposure in the overall dataset as it was exceeded by two other subjects with COPD. The results may suggest that ensifentrine, may be administered to individuals of varying levels of hepatic impairment (e.g., healthy, moderate, or severe hepatic impairment), with similar doses, resulting in similar PK profiles.

### Embodiments

Additional embodiments include the following:
1. A method of treating chronic obstructive pulmonary disease (COPD) in a human subject having moderate renal impairment, the method comprising administering to the human subject a liquid pharmaceutical composition comprising a therapeutically effective amount of ensifentrine or a pharmaceutically acceptable salt thereof.
2. The method of embodiment 1, wherein the human subject has an absolute estimated glomerular filtration rate (aEGFR) of less than 60 mL/min.
3. The method of any one of the preceding embodiments, wherein the liquid pharmaceutical composition provides a blood plasma concentration of ensifentrine after administration by inhalation to the human subject, the blood plasma concentration comprising a mean Cₘₐₓ of from about 500 pg/mL to about 700 pg/mL.
4. The method of embodiment 3, wherein the blood plasma concentration comprises a mean Cₘₐₓ of from about 550 pg/mL to about 650 pg/mL.
5. The method of embodiment 3 or 4, wherein the blood plasma concentration comprises a mean Cₘₐₓ of about 604 pg/mL.
6. The method of any one of embodiments 3-5, wherein the Cₘₐₓ is at least 10% higher than a Cₘₐₓ for a subject administered the liquid pharmaceutical composition by inhalation and not having moderate renal impairment.
7. The method of any one of embodiments 3-6, wherein the blood plasma concentration comprises a mean AUC₀₋ₜₐᵤ of from about 2600 pg/mL*h to about 3600 pg/mL*h.
8. The method of embodiment 7, wherein the blood plasma concentration comprises a mean AUC₀₋ₜₐᵤ of from about 3000 pg/mL*h to about 3400 pg/mL*h.
9. The method of embodiment 7 or 8, wherein the blood plasma concentration comprises a mean AUC₀₋ₜₐᵤ of about 3200 pg/mL*h.
10. The method of any one of embodiments 7-9, wherein the mean AUC₀₋ₜₐᵤ is at least 25% higher than a mean AUC₀₋ₜₐᵤ for a subject administered the liquid pharmaceutical composition by inhalation and not having moderate renal impairment.
11. The method of any one of embodiments 3-10, wherein the blood plasma concentration comprises a mean Tₘₐₓ of from about 1 h to 2 hs.
12. The method of embodiment 11, wherein the blood plasma concentration comprise a mean Tₘₐₓ of from about 1.2 h to about 1.7 h.
13. The method of any one of embodiments 1-12, wherein the mean Cₘₐₓ, mean AUC₀₋ₜₐᵤ, and mean Tₘₐₓ are measured at intervals, wherein the intervals comprise two or more of 1.0 h (±0.5 h), 1.5 h (±0.5 h), 2.5 h (±0.5 h), 3.0 h (±0.5 h), 4.0 h (±1 h), 6.0 h (±1 h), 8.0 h (±1 h), 10.0 h (±1 h), 12.0 h (±1 h), 24.0 h (±1 h), 36.0 h (±1 h), 48.0 h (±1 h), 56.0 h (±1 h) and 60.0 h (±1 h) after administration of the liquid pharmaceutical composition.
14. A method of treating chronic obstructive pulmonary disease (COPD) in a human subject with a liver function comprising healthy liver function, moderate hepatic impairment, or severe hepatic impairment, the method comprising administering to the human subject a liquid pharmaceutical composition comprising a therapeutically effective amount of ensifentrine or a pharmaceutically acceptable salt thereof,
   wherein the liquid pharmaceutical composition provides a blood plasma concentration of ensifentrine comprising an AUC_{t,ss} which is substantially the same for subjects comprising healthy liver function, moderate hepatic impairment, or severe hepatic impairment.
15. The method of embodiment 14, wherein the mean AUC_{t,ss} is of from about 5000 pg/mL*h to about 10000 pg/mL*h.
16. The method of embodiment 14 or 15, wherein the mean AUC_{t,ss} is of from about 5000 pg/mL*h to about 7500 pg/mL*h.
17. The method of any one of embodiments 14-16, wherein the liver function of the human subject is classified using Child Pugh score.
18. The method of any one of embodiments 14-17, wherein the administering is by inhalation.
19. The method of any one of embodiments 3-13 or 18, wherein the inhalation is by nebulizer.
20. The method of any one of the preceding embodiments, wherein the ensifentrine is ensifentrine free base.
21. The method of any one of the preceding embodiments, wherein the administering to the human subject is at least once, twice, or three times per day.
22. The method of any one of the preceding embodiments, wherein the administering to the human subject is at least twice a day.
23. The method of any one of the preceding embodiments, wherein the therapeutically effective amount of ensifentrine or the pharmaceutically acceptable salt thereof is of from about 2 mg to about 4 mg.
24. The method of embodiment 23, wherein the therapeutically effective amount of ensifentrine or the pharmaceutically acceptable salt thereof is 3 mg.
25. The method of any one of the preceding embodiments, wherein the therapeutically effective amount of ensifentrine or the pharmaceutically acceptable salt thereof comprises a total daily dose of the ensifentrine or the pharmaceutically acceptable salt thereof of from about 0.5 mg to about 10 mg.
26. The method of embodiment 25, wherein the total daily dose is of from about 5 mg to about 7 mg.
27. The method of any one of the preceding embodiment, wherein the administering to the human subject comprises a first dose and a second dose per day.
28. The method of embodiment 27, wherein the first dose comprises from about 2 mg to about 4 mg of the ensifentrine or the pharmaceutically acceptable salt thereof and the second dose comprises of from about 2 mg to about 4 mg of ensifentrine or the pharmaceutically acceptable salt thereof.
29. The method of embodiment 27, wherein the first dose and the second dose comprise about 3 mg of the ensifentrine or the pharmaceutically acceptable salt thereof.
30. The method of any one of embodiments 27-29, wherein the first dose is administered in the morning and the second dose is administered in the evening.
31. The method of any one of embodiments 27-29, wherein the first dose is administered within three hours after the human subject waking, and the second dose is administered within three hours before the human subject sleeps.
32. The method of any one of embodiments 27-29, wherein the first dose and the second dose are administered to the human subject about 10 h to about 14 h apart.
33. The method of any one of the preceding embodiments, wherein the administering to the human subject is twice per day for at least 8 weeks.
34. The method of any one of the preceding embodiments, wherein the administering to the human subject is twice per day for at least 16 weeks.
35. The method of any one of the preceding embodiments, wherein the administering to the human subject is twice per day for at least 24 weeks.
36. The method of any one of the preceding embodiments, wherein the liquid pharmaceutical composition comprises a suspension of ensifentrine particles comprising the ensifentrine or a pharmaceutically acceptable salt thereof in a diluent.
37. The method of embodiment 36, wherein the ensifentrine particles comprise at least 95% by weight of the ensifentrine or the pharmaceutically acceptable salt thereof.
38. The method of embodiment 36, wherein the ensifentrine particles comprise at least 95% by weight of ensifentrine free base.
39. The method of any one of embodiments 36-38, wherein the ensifentrine is in the form of a crystalline solid consisting of greater than 99% by weight of ensifentrine free base,
   at least 95% in the polymorphic form of a thermodynamically stable polymorph (I) of ensifentrine free base having the following structural parameters obtained by single crystal analysis:

| Wavelength | 0.71073 Å | |
|---|---|---|
| Crystal system | Triclinic | |
| Space group | P-1 | |
| Unit cell dimensions | a = 8.1246(4) Å | α = 91.583(4)° |
| | b = 11.4573(5) Å | β = 90.299(4)° |
| | c = 13.2398(6) Å | γ = 99.628(4)° |
| Volume | 1214.56(10) Å³ | |
| Z | 2 | |

40. The method of any one of embodiments 36-39, wherein the ensifentrine particles comprise from about 0% to about 0.6% by weight of 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU) relative to total weight of the ensifentrine or the pharmaceutically acceptable salt thereof.
41. The method of any one of embodiments 36-40, wherein the ensifentrine particles comprise from about 0% to about 0.5% by weight of a biuret impurity of formula (A) relative to total weight of the ensifentrine or the pharmaceutically acceptable salt thereof:

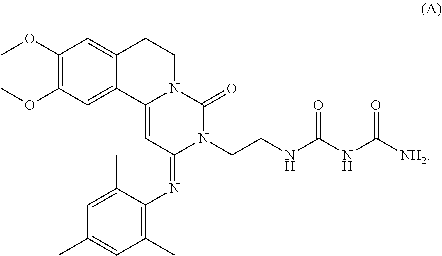 [SMILES: COc1cc2c(cc1OC)-c1c/c(=N\c3c(C)cc(C)cc3C)n(CCNC(=O)NC(N)=O)c(=O)n1CC2]

42. The method of embodiment 41, wherein the ensifentrine particles comprise from about 0% to about 0.3% by weight of the biuret impurity relative to the total weight of the ensifentrine of the pharmaceutically acceptable salt thereof.
43. The method of any one of embodiments 40-42, wherein the ensifentrine particles comprise from about 0.01% to about 0.3% by weight of the BMIQU relative to the total weight of the ensifentrine or the pharmaceutically acceptable salt thereof.
44. The method of any one of embodiments 36-43, wherein the liquid pharmaceutical composition comprises:
   a. ensifentrine particles;
   b. a buffer;
   c. one or more surfactants; and
   d. a tonicity adjuster.
45. The method of any one of embodiments 36-44, wherein the liquid pharmaceutical composition comprises:
   a. ensifentrine particles at a concentration of from about 1 mg/mL to about 1.4 mg/mL;
   b. a buffer at a concentration of from about 1 mg/mL to about 2 mg/mL;
   c. one or more surfactants at a total concentration of from about 0.01 mg/mL to about 0.8 mg/mL; and
   d. a tonicity adjuster at a concentration of from about 5 mg/mL to about 10 mg/mL.
46. The method of any one of embodiments 36-45, wherein the liquid pharmaceutical composition comprises:
   a. ensifentrine particles at a concentration of from about 1 mg/mL to about 1.4 mg/mL;
   b. polysorbate 20 (Tween 20) at a concentration of from about 0.3 mg/mL to about 0.7 mg/mL;
   c. sorbitan monolaurate (Span 20) at a concentration of from 0 mg/mL to about 0.1 mg/mL;
   d. sodium dihydrogen phosphate dihydrate at a concentration of from about 0.5 mg/mL to about 1 mg/mL;
   e. disodium hydrogen phosphate dihydrate at a concentration of from about 0.5 mg/mL to about 1 mg/mL; and
   f. sodium chloride at a concentration of from about 5 mg/mL to about 10 mg/mL.
47. The method of any one of embodiments 36-46, wherein the liquid pharmaceutical composition comprises:
   a. 1.2 mg/mL ensifentrine particles;
   b. 0.5 mg/mL polysorbate 20;
   c. 0.05 mg/mL sorbitan monolaurate;
   d. 0.744 mg/mL sodium dihydrogen phosphate dihydrate;
   e. 0.853 mg/mL disodium hydrogen phosphate dihydrate;
   f. 8.6 mg/mL sodium chloride; and
   g. water.
48. The method of any one of embodiments 36-46, wherein the liquid pharmaceutical composition is a suspension comprising:
   a. 1.4 mg/mL ensifentrine particles;
   b. 0.55 mg/ml polysorbate 20;
   c. 0.744 mg/ml sodium dihydrogen phosphate dihydrate;
   d. 0.853 mg/ml disodium hydrogen phosphate dihydrate;
   e. 8.6 mg/ml sodium chloride; and
   f. water.
49. The method of any one of embodiments 36-46, wherein the liquid pharmaceutical composition is a suspension comprising:
   a. 1.3 mg/mL ensifentrine particles;
   b. 0.55 mg/ml sorbitan monolaurate;
   c. 0.744 mg/ml sodium dihydrogen phosphate dihydrate;
   d. 0.853 mg/ml disodium hydrogen phosphate dihydrate;
   e. 8.6 mg/ml sodium chloride; and
   f. water.
50. The method of any one of embodiments 36-46, wherein the liquid pharmaceutical composition is a suspension comprising:
   a. 1.3 mg/mL ensifentrine particles;
   b. 0.5 mg/ml polysorbate 20;
   c. 0.05 mg/ml sorbitan monolaurate;
   d. 1.1 mg/ml sodium dihydrogen phosphate dihydrate;
   e. 0.9 mg/ml disodium hydrogen phosphate dihydrate;
   f. 13 mg/ml sodium chloride; and
   g. water.
51. The method of any one of embodiments 36-46, wherein the liquid pharmaceutical composition is a suspension comprising:
   a. 1.2 mg/mL ensifentrine particles;
   b. 0.4 mg/ml polysorbate 20;
   c. 0.10 mg/ml sorbitan monolaurate;
   d. 0.744 mg/ml sodium dihydrogen phosphate dihydrate;
   e. 0.853 mg/ml disodium hydrogen phosphate dihydrate;
   f. 8.6 mg/ml sodium chloride; and
   g. water.
52. The method of any one of embodiments 36-46, wherein the liquid pharmaceutical composition is a suspension comprising:
   a. 1.4 mg/mL ensifentrine particles;
   b. 0.4 mg/ml polysorbate 20;
   c. 0.04 mg/ml sorbitan monolaurate;
   d. 1.2 mg/ml sodium dihydrogen phosphate dihydrate;
   e. 8.6 mg/ml sodium chloride; and
   f. water.
53. The method of any one of embodiments 36-46, wherein the liquid pharmaceutical composition comprises:
   a. the ensifentrine particles at a concentration of from 0.8 to 1.4 mg/mL;
   b. one or more surfactants at a total concentration of from 0.3 to 0.7 mg/mL;
   c. one or more buffers at a total concentration of from 1.0 to 2.0 mg/ml;
   d. one or more tonicity adjusters at a concentration of from 1.0 to 15.0 mg/ml and
   e. water.
54. The method of any one of embodiments 45-53, wherein a total weight of the ensifentrine or the pharmaceutically acceptable salt thereof in the liquid pharmaceutical composition is of from about 2.7 mg to about 3.3 mg.
55. The method of any one of the preceding embodiments, wherein the ensifentrine or the pharmaceutically acceptable salt thereof is used in combination with a muscarinic receptor antagonist, a beta-adrenergic receptor agonist, or an inhaled corticosteroid.
56. The method of any one of the preceding embodiments, wherein the COPD is moderate COPD.
57. The method of any one of embodiments 1-55, wherein the COPD is severe COPD.
58. Use of the method of any one of embodiments 1-57 for treating chronic obstructive pulmonary disease (COPD) in a human subject.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

We claim:

1. A method of treating chronic obstructive pulmonary disease (COPD) in a human subject having moderate renal impairment, the method comprising administering to the human subject a liquid pharmaceutical composition comprising a therapeutically effective amount of ensifentrine or a pharmaceutically acceptable salt thereof, wherein the administering to the human subject is at least once, twice, or three times per day.

2. The method of claim 1, wherein the human subject has an absolute estimated glomerular filtration rate (aEGFR) of less than 60 mL/min.

3. The method of claim 1, wherein the liquid pharmaceutical composition provides a blood plasma concentration of ensifentrine after administration by inhalation to the human subject, the blood plasma concentration comprising a mean Cₘₐₓ of from about 500 pg/mL to about 700 pg/mL.

4. The method of claim 3, wherein the Cₘₐₓ is at least 10% higher than a Cₘₐₓ for a subject administered the liquid pharmaceutical composition by inhalation and not having moderate renal impairment.

5. The method of claim 3, wherein the blood plasma concentration comprises a mean AUC₀₋ₜₐᵤ of from about 2600 pg/mL*h to about 3600 pg/mL*h.

6. The method of claim 3, wherein the blood plasma concentration comprises a mean Tₘₐₓ of from about 1 h to 2 hs.

7. The method of claim 1, wherein the therapeutically effective amount of ensifentrine or the pharmaceutically acceptable salt thereof is of from about 2 mg to about 4 mg.

8. The method of claim 1, wherein the therapeutically effective amount of ensifentrine or the pharmaceutically acceptable salt thereof comprises a total daily dose of the ensifentrine or the pharmaceutically acceptable salt thereof of from about 0.5 mg to about 10 mg.

9. The method of claim 1, wherein a first dose comprises from about 2 mg to about 4 mg of the ensifentrine or the pharmaceutically acceptable salt thereof and a second dose comprises of from about 2 mg to about 4 mg of ensifentrine or the pharmaceutically acceptable salt thereof.

10. The method of claim 8, wherein the first dose is administered within three hours after the human subject waking, and the second dose is administered within three hours before the human subject sleeps.

11. The method of claim 10, wherein the first dose and the second dose are administered to the human subject about 10 h to about 14 h apart.

12. The method of claim 1, wherein the administering to the human subject is twice per day for at least 8 weeks, at least 16 weeks, or at least 24 weeks.

13. The method of claim 1, wherein the liquid pharmaceutical composition comprises a suspension of ensifentrine particles comprising the ensifentrine or a pharmaceutically acceptable salt thereof in a diluent.

14. The method of claim 13, wherein the ensifentrine particles comprise at least 95% by weight of the ensifentrine or the pharmaceutically acceptable salt thereof.

15. The method claim 14, wherein the ensifentrine particles comprise from about 0% to about 0.6% by weight of 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU) relative to total weight of the ensifentrine or the pharmaceutically acceptable salt thereof.

16. The method of claim 14, wherein the ensifentrine particles comprise from about 0% to about 0.5% by weight of a biuret impurity of formula (A) relative to total weight of the ensifentrine or the pharmaceutically acceptable salt thereof:

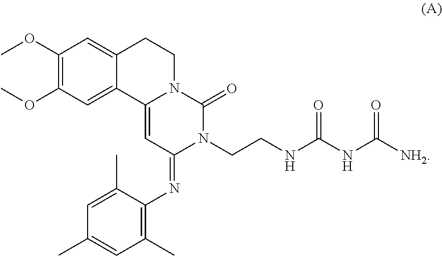 [SMILES: COc1cc2c(cc1OC)-c1c/c(=N\c3c(C)cc(C)cc3C)n(CCNC(=O)NC(N)=O)c(=O)n1CC2]

17. The method of claim 13, wherein the liquid pharmaceutical composition comprises:
a. ensifentrine particles;
b. a buffer;
c. one or more surfactants; and
d. a tonicity adjuster,
wherein a total weight of the ensifentrine or the pharmaceutically acceptable salt thereof in the liquid pharmaceutical composition is of from about 2.7 mg to about 3.3 mg.

18. The method of claim 17, wherein the liquid pharmaceutical composition comprises:
a. ensifentrine particles at a concentration of from about 1 mg/mL to about 1.4 mg/mL;
b. a buffer at a concentration of from about 1 mg/mL to about 2 mg/mL;
c. one or more surfactants at a total concentration of from about 0.01 mg/mL to about 0.8 mg/mL; and
d. a tonicity adjuster at a concentration of from about 5 mg/mL to about 10 mg/mL.

19. The method of claim 17, wherein the liquid pharmaceutical composition comprises:
a. ensifentrine particles at a concentration of from about 1 mg/mL to about 1.4 mg/mL;
b. polysorbate 20 (Tween 20) at a concentration of from about 0.3 mg/mL to about 0.7 mg/mL;
c. sorbitan monolaurate (Span 20) at a concentration of from 0 mg/mL to about 0.1 mg/mL;
d. sodium dihydrogen phosphate dihydrate at a concentration of from about 0.5 mg/mL to about 1 mg/mL;
e. disodium hydrogen phosphate dihydrate at a concentration of from about 0.5 mg/mL to about 1 mg/mL; and
f. sodium chloride at a concentration of from about 5 mg/mL to about 10 mg/mL.

20. The method of claim 17, wherein the liquid pharmaceutical composition comprises:
a. 1.2 mg/mL ensifentrine particles;
b. 0.5 mg/mL polysorbate 20;
c. 0.05 mg/mL sorbitan monolaurate;
d. 0.744 mg/mL sodium dihydrogen phosphate dihydrate;
e. 0.853 mg/mL disodium hydrogen phosphate dihydrate;
f. 8.6 mg/mL sodium chloride; and
g. water.

21. The method of claim 17, wherein the liquid pharmaceutical composition is a suspension comprising:
a. 1.4 mg/mL ensifentrine particles;
b. 0.55 mg/ml polysorbate 20;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

22. The method of claim 17, wherein the liquid pharmaceutical composition is a suspension comprising:
a. 1.3 mg/mL ensifentrine particles;
b. 0.55 mg/ml sorbitan monolaurate;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

23. The method of claim 17, wherein the liquid pharmaceutical composition is a suspension comprising:
a. 1.3 mg/mL ensifentrine particles;
b. 0.5 mg/ml polysorbate 20;
c. 0.05 mg/ml sorbitan monolaurate;
d. 1.1 mg/ml sodium dihydrogen phosphate dihydrate;
e. 0.9 mg/ml disodium hydrogen phosphate dihydrate;
f. 13 mg/ml sodium chloride; and
g. water.

24. The method of claim 17, wherein the liquid pharmaceutical composition is a suspension comprising:
a. 1.2 mg/mL ensifentrine particles;
b. 0.4 mg/ml polysorbate 20;
c. 0.10 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.

25. The method of claim 17, wherein the liquid pharmaceutical composition is a suspension comprising:
a. 1.4 mg/mL ensifentrine particles;
b. 0.4 mg/ml polysorbate 20;
c. 0.04 mg/ml sorbitan monolaurate;
d. 1.2 mg/ml sodium dihydrogen phosphate dihydrate;
e. 8.6 mg/ml sodium chloride; and
f. water.

26. The method of claim 17, wherein the liquid pharmaceutical composition comprises:
a. the ensifentrine particles at a concentration of from 0.8 to 1.4 mg/mL;
b. one or more surfactants at a total concentration of from 0.3 to 0.7 mg/mL;
c. one or more buffers at a total concentration of from 1.0 to 2.0 mg/ml;
d. one or more tonicity adjusters at a concentration of from 1.0 to 15.0 mg/ml and
e. water.

27. The method of claim 1, wherein the COPD is moderate COPD or severe COPD.

28. The method of claim 1, wherein the ensifentrine or the pharmaceutically acceptable salt thereof is used in combination with a muscarinic receptor antagonist, a beta-adrenergic receptor agonist, or an inhaled corticosteroid.

29. A method of treating chronic obstructive pulmonary disease (COPD) in a human subject with a liver function comprising healthy liver function, moderate hepatic impairment, or severe hepatic impairment, the method comprising administering to the human subject a liquid pharmaceutical composition comprising a therapeutically effective amount of ensifentrine or a pharmaceutically acceptable salt thereof,
wherein the liquid pharmaceutical composition provides a blood plasma concentration of ensifentrine comprising an AUC_{t,ss} which is substantially the same for subjects comprising healthy liver function, moderate hepatic impairment, or severe hepatic impairment.

30. The method of claim 29, wherein the mean AUC_{t,ss} is of from about 5000 pg/mL*h to about 10000 pg/mL*h.
